(19) United States
(12) Patent Application Publication
(10) Pub. No.: US 2023/0287360 A1
(43) Pub. Date: Sep. 14, 2023

(54) KETOREDUCTASE POLYPEPTIDES AND POLYNUCLEOTIDES

(71) Applicant: Codexis, Inc., Redwood City, CA (US)

(72) Inventors: Jack Liang, South San Francisco, CA (US); Nandhitha Subramanian, Cambridge (GB); Charlene Ching, San Jose, CA (US); David William Homan, San Ramon, CA (US); Katie Whalen, Charlottesville, VA (US); Matthew Blake Jones, Portage, MI (US)

(21) Appl. No.: 17/761,126

(22) PCT Filed: Sep. 24, 2020
(86) PCT No.: PCT/US2020/052396

§ 371 Date: Mar. 16, 2022

Related U.S. Application Data
(60) Provisional application No. 62/906,268, filed on Sep. 26, 2019.

(51) Int. Cl. C12N 9/04

(52) U.S. Cl. CPC C12N 9/0006; C12Y 101/01184

(57) ABSTRACT

The present invention provides engineered ketoreductase enzymes having improved properties as compared to a naturally occurring wild-type ketoreductase enzyme, as well as polynucleotides encoding the engineered ketoreductase enzymes, host cells capable of expressing the engineered ketoreductase enzymes, and methods of using the engineered ketoreductase enzymes to synthesize a chiral alcohol. The present invention further provides methods of using the engineered enzymes.

The present application claims priority to U.S. Prov. Pat. Appln. Ser. No. 62/906,268, filed Sep. 26, 2019, which is incorporated by reference in its entirety, for all purposes.

### REFERENCE TO SEQUENCE LISTING, TABLE OR COMPUTER PROGRAM

The Sequence Listing concurrently submitted herewith under 37 C.F.R. §1.821 in a computer readable form (CRF) via EFS-Web as file name CX8-195WO2_ST25.txt is herein incorporated by reference. The electronic copy of the Sequence Listing was created on Sep. 23, 2020, with a file size of 664 kilobytes.

### FIELD OF THE INVENTION

The present invention provides engineered ketoreductase enzymes having improved properties as compared to a naturally occurring wild-type ketoreductase enzyme, as well as polynucleotides encoding the engineered ketoreductase enzymes, host cells capable of expressing the engineered ketoreductase enzymes, and methods of using the engineered ketoreductase enzymes.

### BACKGROUND

Enzymes belonging to the ketoreductase (KRED) or carbonyl reductase class (EC1.1.1.184) are useful for the synthesis of optically active alcohols from the corresponding prochiral ketone substrate and by stereoselective reduction of corresponding racemic aldehyde substrates. KREDs typically convert ketone and aldehyde substrates to the corresponding alcohol product, but may also catalyze the reverse reaction, oxidation of an alcohol substrate to the corresponding ketone/aldehyde product. The reduction of ketones and aldehydes and the oxidation of alcohols by enzymes such as KRED requires a co-factor, most commonly reduced nicotinamide adenine dinucleotide (NADH) or reduced nicotinamide adenine dinucleotide phosphate (NADPH), and nicotinamide adenine dinucleotide (NAD+) or nicotinamide adenine dinucleotide phosphate (NADP+) for the oxidation reaction. NADH and NADPH serve as electron donors, while NAD+ and NADP+ serve as electron acceptors. It is frequently observed that ketoreductases and alcohol dehydrogenases accept either the phosphorylated or the non-phosphorylated co-factor (in its oxidized and reduced state), but most often not both.

In order to circumvent many chemical synthetic procedures for the production of key compounds, ketoreductases are increasingly being employed for the enzymatic conversion of different keto and aldehyde substrates to chiral alcohol products. These applications can employ whole cells expressing the ketoreductase for biocatalytic ketone and aldehyde reductions or for biocatalytic alcohol oxidation, or by use of purified enzymes in those instances where presence of multiple ketoreductases in whole cells would adversely affect the stereopurity and yield of the desired product.

“Bitterness” is a key tasting attribute of beer that is typically derived from the addition of hops (flowers of the plant Humulus lupulus L.). Iso-α-acids are formed during the brewing process by the isomerization of the humulones, which are naturally occurring compounds in the lupulin glands of the hop plant. Specifically, the six major iso-α-acids are responsible for the bitter taste: cis-isohumulone, trans-isohumulone, cis-isocohumulone, trans-isocohumulone, cis-isoadhumulone, and trans-isoadhumulone.

However, the iso-α-acids are not light stable, and light-induced formation of 3-methyl-2-butene-1-thiol (3-MBT) gives beer a pronounced light-struck or skunky flavor and aroma. This necessitates the packing of beer in brown bottles or cans. Another solution is to create fully light stable beers by reduction of a carbonyl group of the iso-α-acid to produce the corresponding dihydro-(rho)-iso-α-acid. These reduced dihydro-(rho)-iso-α-acids are stable and can be bottled in clear or green bottles.

However, currently, iso-α-acids can only be converted to dihydro-(rho)-iso-α-acids using toxic, dangerous and non-food grade chemicals (e.g. sodium borohydride). A safe and food-grade conversion of iso-α-acids to dihydro-(rho)-iso-α-acids would, therefore, be of considerable commercial value.

### SUMMARY OF THE INVENTION

The present invention provides engineered ketoreductase enzymes having improved properties as compared to a naturally occurring wild-type ketoreductase enzyme, as well as polynucleotides encoding the engineered ketoreductase enzymes, host cells capable of expressing the engineered ketoreductase enzymes, and methods of using the engineered ketoreductase enzymes.

The present invention provides engineered ketoreductase (“KRED”) enzymes with improved enzymatic activity in the conversion of iso-α-acids to the corresponding dihydro-(rho)-iso-α-acids compared to the naturally-occurring, wild-type ketoreductase from Lactobacillus kefir (SEQ ID NO: 2) or when compared with other engineered ketoreductase enzymes, including the engineered ketoreductase polypeptides of SEQ ID NO: 4, 6, 80, 104, 172, 186, 194, 252, 270, 272, 286, 328, and/or 330.

In some further embodiments, the engineered enzymes have one or more improved properties in addition to improved enzymatic activity. Improvements in enzyme properties include, but are not limited to improved activity across a range of subtrates, improved activity at high substrate concentration, and improved activity at low cofactor concentration.

The present invention provides engineered ketoreductase variants having at least 85%, 90%, 91%, 92%, 93%, 94%, 95%, 96%, 97%, 98%, 99% or more sequence identity to SEQ ID NO: 4, 6, 80, 104, 172, 186, 194, 252, 270, 272, 286, 328, and/or 330.

The present invention also provides engineered ketoreductase variants having at least 85%, 90%, 91%, 92%, 93%, 94%, 95%, 96%, 97%, 98%, 99% or more sequence identity to SEQ ID NO: 4, and at least one substitution or substitution set at one or more positions selected from positions 12, 21, 87, 93, 97, 110, 145, 148, 152, 153, 194, 196, 197, 200, 206, 212, and 226, wherein the positions are numbered with reference to SEQ ID NO: 4. In some additional embodiments, the engineered ketoreductase variants comprise at least one substitution or substitution set selected from 12I, 21R, 87L, 93D, 93M, 93T, 93V, 97G, 110I, 145C, 145G, 145M, 145S, 148I, 152G, 152S, 153C, 153R, 153V, 194H, 194N, 194R, 196H, 196K, 196R, 197G, 197R, 200L, 200Q, 200R, 206V, 212S, and 226L, wherein the positions are numbered with reference to SEQ ID NO: 4. In some further embodiments, the engineered ketoreductase variants comprise at least one substitution or substitution set selected from V12I, L21R, V87L, I93D, I93M, I93T, I93V, K97G, L110I, L145C, L145G, L145M, L145S, V148I, T152G, T152S, L153C, L153R, L153V, P194H, P194N, P194R, L196H, L196K, L196R, D197G, D197R, E200L, E200Q, E200R, M206V, T212S, and I226L, wherein the positions are numbered with reference to SEQ ID NO: 4.

The present invention also provides engineered ketoreductase variants having at least 85%, 90%, 91%, 92%, 93%, 94%, 95%, 96%, 97%, 98%, 99% or more sequence identity to SEQ ID NO: 6, and at least one substitution or substitution set at one or more positions selected from positions 12/110/145/152, 12/145, 87/110/145, 87/110/145/194, 87/145/194, 110, 110/145/152/197, 110/145/194, 145, 145/152, 145/197/226, and 152, wherein the positions are numbered with reference to SEQ ID NO: 6. In some embodiments, the engineered ketoreductase variants comprise at least one substitution or substitution set selected from 12I/110I/145M/152G, 12I/145M, 87L/110I/145M, 87L/110I/145M/194H, 87L/110I/145M/194N, 87L/145M/194H, 110I, 110I/145M/152G/197G, 110I/145M/194H, 145M, 145M/152G, 145M/197G/226L, and 152S, wherein the positions are numbered with reference to SEQ ID NO: 6. In some additional embodiments, the engineered ketoreductase variants comprise at least one substitution or substitution set selected from V12I/L110I/L145M/T152G, V12I/L145M, V87L/L110I/L145M, V87L/L110I/L145M/P194H, V87L/L110I/L145M/P194N, V87L/L145M/P194H, L110I, L110I/L145M/T152G/D197G, L110I/L145M/P194H, L145M, L145M/T152G, L145M/D197G/I226L, and T152S, wherein the positions are numbered with reference to SEQ ID NO: 6.

The present invention also provides engineered ketoreductase variants having at least 85%, 90%, 91%, 92%, 93%, 94%, 95%, 96%, 97%, 98%, 99% or more sequence identity to SEQ ID NO: 80, and at least one substitution or substitution set at one or more positions selected from positions 17, 21, 46, 56, 72, 79, 95, 101, 110, 152, 162, 190, 198, 210, 211, and 227, wherein the positions are numbered with reference to SEQ ID NO: 80. In some additional embodiments, the engineered ketoreductase variants comprise at least one substitution or substitution set selected from 17Q, 17S, 21A, 46V, 56C, 72A, 79L, 95I, 101C, 101L, 101T, 110V, 152K, 152L, 162G, 190A, 198A, 198Q, 210F, 210W, 211R, and 227V, wherein the positions are numbered with reference to SEQ ID NO: 80. In some further embodiments, the engineered ketoreductase variants comprise at least one substitution or substitution set selected from L17Q, L17S, L21A, K46V, V56C, K72A, E79L, V95I, D101C, D101L, D101T, I110V, T152K, T152L, A162G, P190A, D198A, D198Q, T210F, T210W, L211R, and C227V, wherein the positions are numbered with reference to SEQ ID NO: 80.

The present invention also provides engineered ketoreductase variants having at least 85%, 90%, 91%, 92%, 93%, 94%, 95%, 96%, 97%, 98%, 99% or more sequence identity to SEQ ID NO: 80, and at least one substitution or substitution set at one or more positions selected from positions 17, 79, 157, 159, 190/191/194, 190/194, 191/194, 194, 198, and 211, wherein the positions are numbered with reference to SEQ ID NO: 80. In some additional embodiments, the engineered ketoreductase variants comprise at least one substitution or substitution set selected from 17M, 17Q, 17S, 79L, 157C, 159T, 190A/191T/194E, 190A/194E, 191T/194E, 194E, 198A, 198Q, and 211R, wherein the positions are numbered with reference to SEQ ID NO: 80. In some further embodiments, the engineered ketoreductase variants comprise at least one substitution or substitution set selected from L17M, L17Q, L17S, E79L, N157C, S159T, P190A/I191T/P194E, P190A/P194E, I191T/P194E, P194E, D198A, D198Q, and L211R, wherein the positions are numbered with reference to SEQ ID NO: 80.

The present invention also provides engineered ketoreductase variants having at least 85%, 90%, 91%, 92%, 93%, 94%, 95%, 96%, 97%, 98%, 99% or more sequence identity to SEQ ID NO: 104, and at least one substitution or substitution set at one or more positions selected from positions 17/46/190, 17/46/198/211, 17/96/194/198, 17/190/198, 46/190/194/198, and 46/194/198, wherein the positions are numbered with reference to SEQ ID NO: 104. In some additional embodiments, the engineered ketoreductase variants comprise at least one substitution or substitution set selected from 17M/46V/190A, 17M/46V/198A/211R, 17M/96V/194E/198Q, 17M/190A/198A, 17M/190A/198Q, 46V/190A/194E/198Q, and 46V/194E/198Q, wherein the positions are numbered with reference to SEQ ID NO: 104. In some further embodiments, the engineered ketoreductase variants comprise at least one substitution or substitution set selected from Q17M/K46V/P190A, Q17M/K46V/D198A/L211R, Q17M/I96V/P194E/D198Q, Q17M/P190A/D198A, Q17M/P190A/D198Q, K46V/P190A/P194E/D198Q, and K46V/P194E/D198Q, wherein the positions are numbered with reference to SEQ ID NO: 104.

The present invention also provides engineered ketoreductase variants having at least 85%, 90%, 91%, 92%, 93%, 94%, 95%, 96%, 97%, 98%, 99% or more sequence identity to SEQ ID NO: 172, and at least one substitution or substitution set at one or more positions selected from positions 45, 101, 179, 194, 204, 226, and 231, wherein the positions are numbered with reference to SEQ ID NO: 172. In some additional embodiments, the engineered ketoreductase variants comprise at least one substitution or substitution set selected from 45L, 101R, 101Y, 179M, 194E, 204Q, 226V, and 231G, wherein the positions are numbered with reference to SEQ ID NO: 172. In some further embodiments, the engineered ketoreductase variants comprise at least one substitution or substitution set selected from E45L, D101R, D101Y, Y179M, P194E, E204Q, I226V, and A231G, wherein the positions are numbered with reference to SEQ ID NO: 172.

The present invention also provides engineered ketoreductase variants having at least 85%, 90%, 91%, 92%, 93%, 94%, 95%, 96%, 97%, 98%, 99% or more sequence identity to SEQ ID NO: 186, and at least one substitution or substitution set at one or more positions selected from positions 95/96/97/150/153/205, 95/96/150/153/205/206/211/249, 95/97/143/145/150/153/202/205, 95/97/143/145/150/153/249, 95/97/150/153, 95/97/150/153/202/205/206, 95/150/153/205/206/211, 95/150/153/205/211, 95/150/153/206/249, 96/150/153, 96/150/153/206, 97/150/153, 97/150/153/205, 97/150/153/205/211, 97/150/153/206, 143/144/145/150/153/202/205/249, 143/145/150/153, 144/145/150/153/205/206, 144/150/153, 144/150/153/202/205/206, 145/150/153/206/249, 145/153/211, 150/153/202/206/249, 150/153/205/211, 150/153/206/211, 150/153/211, and 150/153/249, wherein the positions are numbered with reference to SEQ ID NO: 186. In some additional embodiments, the engineered ketoreductase variants comprise at least one substitution or substitution set selected from 95A/96A/97A/150A/153A/205A, 95A/96A/150A/153A/205A/206A/211A/249A, 95A/97A/143A/145A/150A/153A/202A/205A, 95A/97A/143A/145A/150A/153A/249A, 95A/97A/150A/153A, 95A/97A/150A/153A/202A/205A/206A, 95A/150A/153A/205A/206A/211A, 95A/150A/153A/205A/211A, 95A/150A/153A/206A/249A, 96A/150A/153A, 96A/150A/153A/206A, 97A/150A/153A, 97A/150A/153A/205A, 97A/150A/153A/205A/211A, 97A/150A/153A/206A, 143A/144A/145A/150A/153A/202A/205A/249A, 143A/145A/150A/153A, 144A/145A/150A/153A/205A/206A, 144A/150A/153A, 144A/150A/153A/202A/205A/206A, 145A/150A/153A/206A/249A, 145A/153A/211A, 150A/153A/202A/206A/249A, 150A/153A/205A/211A, 150A/153A/206A/211A, 150A/153A/211A, and 150A/153A/249A, wherein the positions are numbered with reference to SEQ ID NO: 186. In some further embodiments, the engineered ketoreductase variants comprise at least one substitution or substitution set selected from V95A/I96A/K97A/D150A/L153A/M205A, V95A/I96A/D150A/L153A/M205A/M206A/L211A/W249A, V95A/K97A/S143A/M145A/D150A/L153A/W202A/M205A, V95A/K97A/S143A/M145A/D150A/L153A/W249A, V95A/K97A/D150A/L153A, V95A/K97A/D150A/L153A/W202A/M205A/M206A, V95A/D150A/L153A/M205A/M206A/L211A, V95A/D150A/L153A/M205A/L211A, V95A/D150A/L153A/M206A/W249A, I96A/D150A/L153A, I96A/D150A/L153A/M206A, K97A/D150A/L153A, K97A/D150A/L153A/M205A, K97A/D150A/L153A/M205A/L211A, K97A/D150A/L153A/M206A, S143A/I144A/M145A/D150A/L153A/W202A/M205A/W249A, S143A/M145A/D150A/L153A, I144A/M145A/D150A/L153A/M205A/M206A, I144A/D150A/L153A, I144A/D150A/L153A/W202A/M205A/M206A, M145A/D150A/L153A/M206A/W249A, M145A/L153A/L211A, D150A/L153A/W202A/M206A/W249A, D150A/L153A/M205A/L211A, D150A/L153A/M206A/L211A, D150A/L153A/L211A, and D150A/L153A/W249A, wherein the positions are numbered with reference to SEQ ID NO: 186.

The present invention also provides engineered ketoreductase variants having at least 85%, 90%, 91%, 92%, 93%, 94%, 95%, 96%, 97%, 98%, 99% or more sequence identity to SEQ ID NO: 186, and at least one substitution or substitution set at one or more positions selected from positions 7/147, 103/147, 110, 110/179/194, 147, and 249, wherein the positions are numbered with reference to SEQ ID NO: 186. In some additional embodiments, the engineered ketoreductase variants comprise at least one substitution or substitution set selected from 7Q/147I, 103R/147I, 110V, 110V/179M/194E, 147I, and 249Y, wherein the positions are numbered with reference to SEQ ID NO: 186. In some further embodiments, the engineered ketoreductase variants comprise at least one substitution or substitution set selected from H7Q/L147I, T103R/L147I, I110V, I110V/Y179M/P194E, L147I, and W249Y, wherein the positions are numbered with reference to SEQ ID NO: 186.

The present invention also provides engineered ketoreductase variants having at least 85%, 90%, 91%, 92%, 93%, 94%, 95%, 96%, 97%, 98%, 99% or more sequence identity to SEQ ID NO: 194, and at least one substitution or substitution set at one or more positions selected from positions 7/12/54/110/150/153/194/205/211/249, 12/54/72/110/150/152/153/194/205/211/249, 12/72/101/103/110/152/249, 12/72/110/147/152/204, 45/54/72/110/152/194/204, 72/110/147/150/152/153/194/205/211/249, and 110/150/153/179/194/205/211/249, wherein the positions are numbered with reference to SEQ ID NO: 194. In some additional embodiments, the engineered ketoreductase variants comprise at least one substitution or substitution set selected from 7Q/12I/54S/110V/150D/153L/194E/205M/211L/249Y, 12I/54S/72T/110V/150D/152M/153L/194E/205M/211L/249Y, 12I/72S/101Y/103Q/110V/152M/249Y, 12I/72S/110V/147I/152M/204Q, 45L/54S/72S/110V/152M/194E/204Q, 72S/110V/147M/150D/152M/153L/194E/205M/211L/249Y, and 110V/150D/153L/179M/194E/205M/211L/249Y, wherein the positions are numbered with reference to SEQ ID NO: 194. In some further embodiments, the engineered ketoreductase variants comprise at least one substitution or substitution set selected from H7Q/V12I/T54S/I110V/A150D/A153L/P194E/A205M/A211L/W249Y, V12I/T54S/K72T/I110V/A150D/T152M/A153L/P194E/A205M/A211L/W249Y, V12I/K72S/R101Y/T103Q/I110V/T152M/W249Y, V12I/K72S/I110V/L147I/T152M/E204Q, E45L/T54S/K72S/I110V/T152M/P194E/E204Q, K72S/I110V/L147M/A150D/T152M/A153L/P194E/A205M/A211L/W249Y, and I110V/A150D/A153L/Y179M/P194E/A205M/A211L/W249Y, wherein the positions are numbered with reference to SEQ ID NO: 194.

The present invention also provides engineered ketoreductase variants having at least 85%, 90%, 91%, 92%, 93%, 94%, 95%, 96%, 97%, 98%, 99% or more sequence identity to SEQ ID NO: 252, and at least one substitution or substitution set at one or more positions selected from positions 7/12/54/179/249, 7/152, 12/54/72/152/179/249, 40, 54/72, 72/147/152/179/249, and 249, wherein the positions are numbered with reference to SEQ ID NO: 252. In some additional embodiments, the engineered ketoreductase variants comprise at least one substitution or substitution set selected from 7Q/12I/54S/179Y/249Y, 7Q/152M, 12I/54S/72T/152M/179Y/249Y, 40E, 54S/72S, 72S/147M/152M/179Y/249Y, and 249Y, wherein the positions are numbered with reference to SEQ ID NO: 252. In some further embodiments, the engineered ketoreductase variants comprise at least one substitution or substitution set selected from H7Q/V12I/T54S/M179Y/W249Y, H7Q/T152M, V12I/T54S/K72T/T152M/M179Y/W249Y, H40E, T54S/K72S, K72S/L147M/T152M/M179Y/W249Y, and W249Y, wherein the positions are numbered with reference to SEQ ID NO: 252.

The present invention also provides engineered ketoreductase variants having at least 85%, 90%, 91%, 92%, 93%, 94%, 95%, 96%, 97%, 98%, 99% or more sequence identity to SEQ ID NO: 270, and at least one substitution or substitution set at one or more positions selected from positions 92/93, 150/152, 150/152/153, and 194/195, wherein the positions are numbered with reference to SEQ ID NO: 270. In some additional embodiments, the engineered ketoreductase variants comprise at least one substitution or substitution set selected from 92A/93E, 150D/152A/153L, 150Y/152A, 150Y/152S, and 194S/195A, wherein the positions are numbered with reference to SEQ ID NO: 270. In some further embodiments, the engineered ketoreductase variants comprise at least one substitution or substitution set selected from G92A/I93E, A150D/M152A/A153L, A150Y/M152A, A150Y/M152S, and E194S/R195A, wherein the positions are numbered with reference to SEQ ID NO: 270.

The present invention also provides engineered ketoreductase variants having at least 85%, 90%, 91%, 92%, 93%, 94%, 95%, 96%, 97%, 98%, 99% or more sequence identity to SEQ ID NO: 272, and at least one substitution or substitution set at one or more positions selected from positions 92/93/95, 93, 93/95, 93/95/109, 93/95/109/114, 93/95/114, 93/109/114, and 114, wherein the positions are numbered with reference to SEQ ID NO: 272. In some additional embodiments, the engineered ketoreductase variants comprise at least one substitution or substitution set selected from 92A/93D/95R, 93A/95K/109R, 93A/95R/109D/114T, 93D/95R, 93E/109R/114A, 93M, 93R/95A/114T, and 114A, wherein the positions are numbered with reference to SEQ ID NO: 272. In some further embodiments, the engineered ketoreductase variants comprise at least one substitution or substitution set selected from G92A/I93D/V95R, I93A/V95K/K109R, I93A/V95R/K109D/N114T, I93D/V95R, I93E/K109R/N114A, I93M, I93R/V95A/N114T, and N114A, wherein the positions are numbered with reference to SEQ ID NO: 272.

The present invention also provides engineered ketoreductase variants having at least 85%, 90%, 91%, 92%, 93%, 94%, 95%, 96%, 97%, 98%, 99% or more sequence identity to SEQ ID NO: 286, and at least one substitution or substitution set at one or more positions selected from positions 12/45/72/109/249, 12/45/93/249, 12/45/249, 12/109/249, 45/72/249, 45/109/249, 45/249, 96, and 145/150, wherein the positions are numbered with reference to SEQ ID NO: 286. In some additional embodiments, the engineered ketoreductase variants comprise at least one substitution or substitution set selected from 12I/45E/72T/109D/249Y, 12I/45E/93A/249Y, 12I/45E/249Y, 12I/109D/249Y, 45E/72T/249Y, 45E/109D/249Y, 45E/249Y, 96A, and 145A/150A, wherein the positions are numbered with reference to SEQ ID NO: 286. In some further embodiments, the engineered ketoreductase variants comprise at least one substitution or substitution set selected from V12I/L45E/S72T/K109D/W249Y, V12I/L45E/I93A/W249Y, V12I/L45E/W249Y, V12I/K109D/W249Y, L45E/S72T/W249Y, L45E/K109D/W249Y, L45E/W249Y, I96A, and M145A/Y150A, wherein the positions are numbered with reference to SEQ ID NO: 286.

The present invention also provides engineered ketoreductase variants having at least 85%, 90%, 91%, 92%, 93%, 94%, 95%, 96%, 97%, 98%, 99% or more sequence identity to SEQ ID NO: 328, and at least one substitution or substitution set at one or more positions selected from positions 150, 150/151, 150/195, and 195, wherein the positions are numbered with reference to SEQ ID NO: 328. In some additional embodiments, the engineered ketoreductase variants comprise at least one substitution or substitution set selected from 150A, 150A/151A, 150A/195S, 195A, and 195S, wherein the positions are numbered with reference to SEQ ID NO: 328. In some further embodiments, the engineered ketoreductase variants comprise at least one substitution or substitution set selected from Y150A, Y150A/P151A, Y150A/R195S, R195A, and R195S, wherein the positions are numbered with reference to SEQ ID NO: 328.

The present invention also provides engineered ketoreductase variants having at least 85%, 90%, 91%, 92%, 93%, 94%, 95%, 96%, 97%, 98%, 99% or more sequence identity to SEQ ID NO: 330, and at least one substitution or substitution set at one or more positions selected from positions 12/72/109/195, 17/73/200, 17/115, 68/72/101/152/205, 68/72/124, 68/72/124/152, 68/101/124/152/205, 68/124/205, 72/109/152/195, 72/109/195, 72/152, 72/152/195, 72/195, 73, 73/147, 79, 93, 93/95/145/195, 93/109/114/145/195, 93/195, 95/195, 96/108/147/200, 96/194/200, 101/205, 145/195, 147, 147/200, 192, 194, 194/200, 195, 198, and 200, wherein the positions are numbered with reference to SEQ ID NO: 330. In some additional embodiments, the engineered ketoreductase variants comprise at least one substitution or substitution set selected from 12V/72S/109K/195R, 17A/73V/200P, 17A/115Q, 68E/72D/101K/152Q/205L, 68R/72D/101Q/152Q/205L, 68R/72R/124E, 68R/72R/124E/152Q, 68R/101Q/124E/152Q/205L, 68R/124E/205L, 72D/152Q, 72 K/152M/195R, 72 K/195R, 72S/109K/152M/195R, 72S/109K/195R, 73V, 73V/147I, 79A, 93A, 93A/95R/145A/195R, 93A/109K/114T/145A/195R, 93A/195R, 95R/195R, 96P/108S/147I/200P, 96P/194N/200P, 101M/205L, 145A/195A, 147I, 147I/200P, 192R, 194N, 194N/200P, 195R, 198G, 198R, and 200P, wherein the positions are numbered with reference to SEQ ID NO: 330. In some further embodiments, the engineered ketoreductase variants comprise at least one substitution or substitution set selected I12V/T72S/D109K/S195R, M17A/L73V/E200P, M17A/L115Q, A68E/T72D/R101K/A152Q/A205L, A68R/T72D/R101Q/A152Q/A205L, A68R/T72R/L124E, A68R/T72R/L124E/A152Q, A68R/R101Q/L124E/A152Q/A205L, A68R/L124E/A205L, T72D/A152Q, T72K/A152M/S195R, T72K/S195R, T72S/D109K/A152M/S195R, T72S/D109K/S195R, L73V, L73V/L147I, E79A, I93A, I93A/V95R/M145A/S195R, I93A/D109K/N114T/M145A/S195R, I93A/S195R, V95R/S195R, I96P/R108S/L147I/E200P, I96P/E194N/E200P, R101M/A205L, M145A/S195A, L147I, L147I/E200P, K192R, E194N, E194N/E200P, S195R, Q198G, Q198R, and E200P, wherein the positions are numbered with reference to SEQ ID NO: 330.

The present invention also provides engineered ketoreductase variants comprising polypeptide sequences comprising sequences having at least 90% sequence identity to SEQ ID NO: 4, 6, 80, 104, 172, 186, 194, 252, 270, 272, 286, 328, and/or 330. In some embodiments, the engineered ketoreductase variants comprise polypeptide sequences comprising sequences having at least 95% sequence identity to SEQ ID NO: 4, 6, 80, 104, 172, 186, 194, 252, 270, 272, 286, 328, and/or 330. In some further embodiments, the engineered ketoreductase variants comprise polypeptide sequences set forth in SEQ ID NO: 4, 6, 80, 104, 172, 186, 194, 252, 270, 272, 286, 328, and/or 330. In some additional embodiments, the engineered ketoreductase variants comprise polypeptide sequences encoding variants provided in Table 5-1, 6-1, 7-1, 8-1, 17-2, 18-1, 19-1, 19-2, 20-1, 20-2, 21-1, 22-1 and/or 24-1. In some further embodiments, the engineered ketoreductase variants comprise polypeptide sequences selected from the even-numbered sequences set forth in SEQ ID NOS: 6 to 412.

The present invention also provides engineered polynucleotide sequences encoding the engineered ketoreductase variants provided herein. In some embodiments, the engineered polynucleotide sequence comprises a polynucleotide sequence that is at least 85%, 90%, 91%, 92%, 93%, 94%, 95%, 96%, 97%, 98%, 99% or more identical to a sequence selected from the odd-numbered sequences set forth in SEQ ID NOS: 5 to 411. The present invention also provides vectors comprising the engineered polynucleotide sequences encoding the engineered ketoreductase variants provided herein. In some embodiments, the vectors further comprise at least one control sequence. In some embodiments, the vectors comprise SEQ ID NO: 413 and/or 414.

The present invention also provides host cells comprising the vectors comprising polynucleotides encoding the engineered ketoreductase variants provided herein.

The present invention also provides methods of producing the engineered ketoreductase variants provided herein, comprising culturing the host cells provided herein under conditions that the engineered ketoreductase variant is produced by the host cell. In some embodiments, the methods further comprise the step of recovering the engineered ketoreductase variant produced by the host cell. In some further embodiments, the methods of producing the engineered ketoreductase variants comprise culturing a host cell comprising the vector of SEQ ID NO: 413 and/or 414.

The present invention also provides immobilized engineered ketoreductase variants.

The present invention further provides compositions comprising at least one engineered ketoreductase variant provided herein. In some embodiments, the compositions comprise at least one immobilized engineered ketoreductase variant provided herein.

### BRIEF DESCRIPTION OF THE FIGURES

FIG. 1 provides a typical HPLC reaction profile comparing the KRED activity of the polypeptides of SEQ ID NO: 6 and SEQ ID NO: 80 at high substrate concentration.

FIG. 2 provides the results of the experiments described in Example 11, KRED activity (% conversion) of selected variants at high substrate and low NADP concentration.

FIG. 3 provides a typical HPLC reaction profile depicting the Rho species produced by the polypeptide of SEQ ID NO:194.

FIG. 4 provides a typical HPLC reaction profile comparing the KRED activity of the polypeptides of SEQ ID NO: 328 and SEQ ID NO: 330 at high substrate and low NADP concentration. For FIG. 4, Solid Lines = 4 g/L enzyme; Dashed lines = 1 g/L enzyme; Diamond = SEQ ID NO: 328; Open Squares = SEQ ID NO: 330.

FIG. 5 provides a typical HPLC reaction profile comparing the KRED activity of the polypeptides of SEQ ID NO:270, SEQ ID NO: 328, SEQ ID NO: 330, SEQ ID NO: 348, SEQ ID NO: 346, and SEQ ID NO: 356 at high substrate and low NADP concentration. For FIG. 5, Open Triangle = SEQ ID NO:270; Filled Triangle = SEQ ID NO: 328; Open Circle= SEQ ID NO: 348; Filled Circle = SEQ ID NO: 356; Open Square = SEQ ID NO: 330; Filled Square = SEQ ID NO: 346.

### DESCRIPTION OF THE INVENTION

The present invention provides engineered ketoreductase enzymes having improved properties as compared to a naturally occurring wild-type ketoreductase, as well as polynucleotides encoding the engineered ketoreductase enzymes, host cells capable of expressing the engineered ketoreductase enzymes, and methods of using the engineered ketoreductase enzymes.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings. All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Indeed, it is intended that the present invention not be limited to the particular methodology, protocols, and reagents described herein, as these may vary, depending upon the context in which they are used. The headings provided herein are not limitations of the various aspects or embodiments of the present invention.

Nonetheless, in order to facilitate understanding of the present invention, a number of terms are defined below. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation and amino acid sequences are written left to right in amino to carboxy orientation, respectively.

The headings provided herein are not limitations of the various aspects or embodiments of the invention that can be had by reference to the specification as a whole. Accordingly, the terms defined below are more fully defined by reference to the specification as a whole.

“Ketoreductase” and “KRED” are used interchangeably herein to refer to a polypeptide having an enzymatic capability of reducing a carbonyl group to its corresponding alcohol. More specifically, the ketoreductase polypeptides of the invention are capable of reducing a mixture of iso-α-acids to the corresponding dihydro-(rho)-iso-α-acids, as shown in Scheme 1. The ketoreductase enzymes of the current invention are derived from the naturally occurring KRED of L. kefir (SEQ ID NO: 2). However, the terms KRED and ketoreductase are not thus limited, and may refer to naturally occurring enzymes or enzymes derived from various species of bacteria, plants, algae, and/or animal species. The enzymes may be synthetic, man-made, or produced by various methods known to those skilled in the art.

“Iso-α-acids” or “iso” are used interchangeably herein to refer to the isomers, epimers, diastereomers, tautomers and enantiomers of isohumulone, a compound derived from hops, the flowers of the hop plant, Humulus lupulus L. The “iso-α-acids” or “iso” include cis-isohumulone, trans-isohumulone, cis-isocohumulone, trans-isocohumulone, cis-isoadhumulone, and trans-isoadhumulone, but are not limited thereto. The “iso-α-acids” or “iso” also include any naturally occurring or synthetic isomers, epimers, diastereomers, tautomers enantiomers or other derivates or similar compounds that have similar chemical properties, specifically conferring a bitter taste or bitterness to beer or other alcoholic or similar beverages. This includes any isomers, epimers, diastereomers, enantiomers or tautomers of the isohumulone tetronic acid core.

“Dihydro-(rho)-iso-α-acids” or “rho” are used interchangeably herein to refer to the compounds created by the reduction of a carbonyl group of an “iso-α-acid” or “iso,” as defined herein. “Dihydro-(rho)-iso-α-acids” or “rho” can be produced from “iso-α-acids” or “iso” through conversion by one or more KRED polypeptides, as described herein.

As used herein, the terms “protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

As used herein, “polynucleotide” and “nucleic acid” refer to two or more nucleosides that are covalently linked together. The polynucleotide may be wholly comprised of ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). Preferably, such modified or synthetic nucleobases will be encoding nucleobases.

As used herein, “coding sequence” refers to that portion of a nucleic acid (e.g., a gene) that encodes an amino acid sequence of a protein.

As used herein, “naturally occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “non-naturally occurring” or “engineered” or “recombinant” when used in the present invention with reference to (e.g., a cell, nucleic acid, or polypeptide), refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, “percentage of sequence identity,” “percent identity,” and “percent identical” refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (See e.g., Altschul et al., J. Mol. Biol. 215: 403-410 [1990]; and Altschul et al., Nucleic Acids Res. 3389-3402 [1977]). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (See e.g., Henikoff and Henikoff, Proc Natl Acad Sci USA 89: 10915 [1989]).

Numerous other algorithms are available and known in the art that function similarly to BLAST in providing percent identity for two sequences. Optimal alignment of sequences for comparison can be conducted using any suitable method known in the art (e.g., by the local homology algorithm of Smith and Waterman, Adv. Appl. Math. 2:482 [1981]; by the homology alignment algorithm of Needleman and Wunsch, J. Mol. Biol. 48:443 [1970]; by the search for similarity method of Pearson and Lipman, Proc. Natl. Acad. Sci. USA 85: 2444 [1988]; and/or by computerized implementations of these algorithms [GAP, BESTFIT, FASTA, and TFASTA in the GCG Wisconsin Software Package]), or by visual inspection, using methods commonly known in the art. Additionally, determination of sequence alignment and percent sequence identity can employ the BESTFIT or GAP programs in the GCG Wisconsin Software package (Accelrys, Madison WI), using the default parameters provided.

As used herein, “reference sequence” refers to a defined sequence to which another sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity. The term “reference sequence” is not intended to be limited to wild-type sequences, and can include engineered or altered sequences. For example, in some embodiments, a “reference sequence” can be a previously engineered or altered amino acid sequence.

As used herein, “comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered ketoreductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. As used herein, a reference to a residue position, such as “Xn” as further described below, is to be construed as referring to “a residue corresponding to”, unless specifically denoted otherwise. Thus, for example, “X94” refers to any amino acid at position 94 in a polypeptide sequence.

As used herein, “stereoselectivity” refers to the preferential formation in a chemical or enzymatic reaction of one stereoisomer over another stereoisomer or another set of stereoisomers. Stereoselectivity can be partial, where the formation of a stereoisomer is favored over another, or it may be complete where only one stereoisomer is formed. When the stereoisomers are enantiomers, the stereoselectivity is referred to as enantioselectivity, the fraction (typically reported as a percentage) of one enantiomer in the sum of both enantiomers. It is commonly alternatively reported in the art (typically as a percentage) as the enantiomeric excess (e.e.) calculated therefrom according to the formula [major enantiomer – minor enantiomer]/[major enantiomer + minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess. It is also to be understood that stereoselectivity is not limited to single stereoisomers and can be described for sets of stereoisomers.

As used herein, “highly stereoselective” refers to a chemical or enzymatic reaction that is capable of converting a substrate to its corresponding chiral alcohol product, with at least about 75% stereomeric excess.

As used herein, “increased enzymatic activity” and “increased activity” refer to an improved property of an engineered enzyme, which can be represented by an increase in specific activity (e.g., product produced/time/weight protein) or an increase in percent conversion of the substrate to the product (e.g., percent conversion of starting amount of substrate to product in a specified time period using a specified amount of ketoreductase) as compared to a reference enzyme. Exemplary methods to determine enzyme activity are provided in the Examples. Any property relating to enzyme activity may be affected, including the classical enzyme properties of Km, Vmax or kcat, changes of which can lead to increased enzymatic activity. The ketoreductase activity can be measured by any one of standard assays used for measuring ketoreductases, such as change in substrate or product concentration, or change in concentration of the cofactor (in absence of a cofactor regenerating system). Comparisons of enzyme activities are made using a defined preparation of enzyme, a defined assay under a set condition, and one or more defined substrates, as further described in detail herein. Generally, when enzymes in cell lysates are compared, the numbers of cells and the amount of protein assayed are determined as well as use of identical expression systems and identical host cells to minimize variations in amount of enzyme produced by the host cells and present in the lysates.

As used herein, “conversion” refers to the enzymatic transformation of a substrate to the corresponding product.

As used herein “percent conversion” refers to the percent of the substrate that is converted to the product within a period of time under specified conditions. Thus, for example, the “enzymatic activity” or “activity” of a ketoreductase polypeptide can be expressed as “percent conversion” of the substrate to the product.

As used herein, “thermostable” or “thermal stable” are used interchangeably to refer to a polypeptide that is resistant to inactivation when exposed to a set of temperature conditions (e.g., 40-80° C.) for a period of time (e.g., 0.5-24 hrs) compared to the untreated enzyme, thus retaining a certain level of residual activity (e.g., more than 60% to 80% for example) after exposure to elevated temperatures.

As used herein, “solvent stable” refers to the ability of a polypeptide to maintain similar activity (e.g., more than e.g., 60% to 80%) after exposure to varying concentrations (e.g., 5-99%) of solvent compared to the untreated enzyme.

As used herein, “amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X40 as compared to SEQ ID NO: 2” refers to a difference of the amino acid residue at the polypeptide position corresponding to position 40 of SEQ ID NO: 2. Thus, if the reference polypeptide of SEQ ID NO: 2 has a histidine at position 40, then a “residue difference at position X40 as compared to SEQ ID NO: 2” refers to an amino acid substitution of any residue other than histidine at the position of the polypeptide corresponding to position 40 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances, the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X192A/G). The present invention includes engineered polypeptide sequences comprising one or more amino acid differences that include either/or both conservative and non-conservative amino acid substitutions. The amino acid sequences of the specific recombinant ketoreductase polypeptides included in the Sequence Listing of the present invention include an initiating methionine (M) residue (i.e., M represents residue position 1). The skilled artisan, however, understands that this initiating methionine residue can be removed by biological processing machinery, such as in a host cell or in vitro translation system, to generate a mature protein lacking the initiating methionine residue, but otherwise retaining the enzyme’s properties. Consequently, the term “amino acid residue difference relative to SEQ ID NO: 2 at position Xn” as used herein may refer to position “Xn” or to the corresponding position (e.g., position (X-1)n) in a reference sequence that has been processed so as to lack the starting methionine.

As used herein, the phrase “conservative amino acid substitutions” refers to the interchangeability of residues having similar side chains, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with a hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1.

TABLE 1

Exemplary Conservative Amino Acid Substitutions

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | Non-polar |

As used herein, the phrase “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As will be appreciated by those of skill in the art, some of the above-defined categories, unless otherwise specified, are not mutually exclusive. Thus, amino acids having side chains exhibiting two or more physico-chemical properties can be included in multiple categories. The appropriate classification of any amino acid or residue will be apparent to those of skill in the art, especially in light of the detailed invention provided herein.

As used herein, “deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

As used herein, “insertion” refers to modification of the polypeptide by addition of one or more amino acids to the reference polypeptide. In some embodiments, the improved engineered ketoreductase enzymes comprise insertions of one or more amino acids to the naturally occurring ketoreductase polypeptide as well as insertions of one or more amino acids to engineered ketoreductase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant KREDs listed in the Tables provided in the Examples.

As used herein, “fragment” refers to a polypeptide that has an amino-terminal and/or carboxy-terminal deletion, but where the remaining amino acid sequence is identical to the corresponding positions in the sequence. Fragments can typically have about 80%, about 90%, about 95%, about 98%, or about 99% of the full-length ketoreductase polypeptide, for example the polypeptide of SEQ ID NO:4. In some embodiments, the fragment is “biologically active” (i.e., it exhibits the same enzymatic activity as the full-length sequence).

As used herein, “isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The improved ketoreductase enzymes may be present within a cell, present in the cellular medium, or prepared in various forms, such as lysates or isolated preparations. As such, in some embodiments, the engineered ketoreductase polypeptides of the present invention can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure engineered ketoreductase polypeptide composition will comprise about 60 % or more, about 70% or more, about 80% or more, about 90% or more, about 91% or more, about 92% or more, about 93% or more, about 94% or more, about 95% or more, about 96% or more, about 97% or more, about 98% or more, or about 99% of all macromolecular species by mole or % weight present in the composition. Solvent species, small molecules (<500 Daltons), and elemental ion species are not considered macromolecular species. In some embodiments, the isolated improved ketoreductase polypeptide is a substantially pure polypeptide composition.

As used herein, when used with reference to a nucleic acid or polypeptide, the term “heterologous” refers to a sequence that is not normally expressed and secreted by an organism (e.g., a wild-type organism). In some embodiments, the term encompasses a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature (e.g., a nucleic acid open reading frame (ORF) of the invention operatively linked to a promoter sequence inserted into an expression cassette, such as a vector). In some embodiments, “heterologous polynucleotide” refers to any polynucleotide that is introduced into a host cell by laboratory techniques, and includes polynucleotides that are removed from a host cell, subjected to laboratory manipulation, and then reintroduced into a host cell.

As used herein, “codon optimized” refers to changes in the codons of the polynucleotide encoding a protein to those preferentially used in a particular organism such that the encoded protein is efficiently expressed in the organism of interest. In some embodiments, the polynucleotides encoding the ketoreductase enzymes may be codon optimized for optimal production from the host organism selected for expression.

As used herein, “control sequence” is defined herein to include all components, which are necessary or advantageous for the expression of a polynucleotide and/or polypeptide of the present invention. Each control sequence may be native or foreign to the polynucleotide of interest. Such control sequences include, but are not limited to, a leader, polyadenylation sequence, propeptide sequence, promoter, signal peptide sequence, and transcription terminator.

As used herein, “operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the phrases “cofactor regeneration system” and “cofactor recycling system” refer to a set of reactants that participate in a reaction that reduces the oxidized form of the cofactor (e.g., NADP+ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. Cofactor regeneration systems to regenerate NADH or NADPH from NAD+ or NADP+, respectively, are known in the art and may be used in the methods described herein.

As used herein, “suitable reaction conditions” refer to those conditions in the biocatalytic reaction solution (e.g., ranges of enzyme loading, substrate loading, cofactor loading, temperature, pH, buffers, co-solvents, etc.) under which ketoreductase polypeptides of the present invention are capable of stereoselectively reducing a substrate compound to a product compound. Exemplary “suitable reaction conditions” are provided in the present invention and illustrated by the Examples.

As used herein, “loading,” such as in “compound loading,” “enzyme loading,” or “cofactor loading” refers to the concentration or amount of a component in a reaction mixture at the start of the reaction.

As used herein, “substrate” in the context of a biocatalyst mediated process refers to the compound or molecule acted on by the biocatalyst. For example, an exemplary substrate for the ketoreductase biocatalyst in the process disclosed herein is an iso-α-acid.

As used herein “product” in the context of a biocatalyst mediated process refers to the compound or molecule resulting from the action of the biocatalyst.

As used herein, “equilibration” as used herein refers to the process resulting in a steady state concentration of chemical species in a chemical or enzymatic reaction (e.g., interconversion of two species A and B), including interconversion of stereoisomers, as determined by the forward rate constant and the reverse rate constant of the chemical or enzymatic reaction.

As used herein, “oxo” refers to =O.

As used herein, “oxy” refers to a divalent group —O—, which may have various substituents to form different oxy groups, including ethers and esters.

As used herein, “carboxy” refers to —COOH.

As used herein, “carbonyl” refers to —C(O)—, which may have a variety of substituents to form different carbonyl groups including acids, acid halides, aldehydes, amides, esters, and ketones.

As used herein, “hydroxy” refers to —OH.

As used herein, “optional” and “optionally” means that the subsequently described event or circumstance may or may not occur, and that the description includes instances where the event or circumstance occurs and instances in which it does not. One of ordinary skill in the art would understand that with respect to any molecule described as containing one or more optional substituents, only sterically practical and/or synthetically feasible compounds are meant to be included.

As used herein, “optionally substituted” refers to all subsequent modifiers in a term or series of chemical groups. For example, in the term “optionally substituted arylalkyl, the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

### Engineered Enzyme Polypeptides

Ketoreductase (KRED) or carbonyl reductase biocatalysts (EC 1.1.1.184) are useful for the synthesis of alcohols from aldehydes and ketones, and optically active secondary alcohols from the corresponding prostereoisomeric ketone substrates. KREDs may also catalyze the reverse reaction, (i.e., oxidation of an alcohol substrate to the corresponding aldehydes/ketone product). The reduction of aldehydes and ketones and the oxidation of alcohols by KREDs uses a co-factor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP+) for the oxidation reaction. NADH and NADPH serve as electron donors, while NAD+ and NADP+ serve as electron acceptors.

KREDs can be found in a wide range of bacteria and yeasts, as known in the art (See e.g., Hummel and Kula Eur. J. Biochem., 184:1-13 [1989]). Numerous KRED genes and enzyme sequences have been reported, including those of Candida magnoliae (Genbank Acc. No. JC7338; GI: 11360538); Candida parapsilosis (Genbank Acc. No. BAA24528.1; GI: 2815409), Sporobolomyces salmonicolor (Genbank Acc. No. AF160799; GI: 6539734), Lactobacillus kefir (Genbank Acc. No. AAP94029.1; GI: 33112056), Lactobacillus brevis (Genbank Acc. No. 1NXQ_A; GI: 30749782), and Thermoanaerobium brockii (Genbank Acc. No. P14941; GI: 1771790).

The stereoselectivity of ketoreductases have been applied to the preparation of important pharmaceutical building blocks (See e.g., Broussy et al., Org. Lett., 11:305-308 [2009]). Specific applications of naturally occurring or engineered KREDs in biocatalytic processes to generate useful chemical compounds have been demonstrated for reduction of 4-chloroacetoacetate esters (See e.g,. Zhou, J. Am. Chem. Soc.,105:5925-5926 [1983]; Santaniello, J. Chem. Res., (S)132-133 [1984]; U.S. Pat. Nos. 5,559,030; U.S. Pat. No. 5,700,670; and U.S. Pat. No. 5,891,685), reduction of dioxocarboxylic acids (See e.g., U.S. Pat. No. 6,399,339), reduction of tert-butyl (S)-chloro-5-hydroxy-3-oxohexanoate (See e.g., U.S. Pat. No. 6,645,746; and WO 01/40450), reduction of pyrrolotriazine-based compounds (See e.g., U.S. Appln. Publ. No. 2006/0286646); reduction of substituted acetophenones (See e.g., U.S. Pat. Nos. 6,800,477 and 8,748,143); and reduction of ketothiolanes (WO 2005/054491).

The iso-α-acids (“iso”) exist as a complex mixture of cis and trans epimers. In all, there are three different side chains (“R” on iso), commonly referred to as n-, ad- and co- (iBu, sBu, and iPr respectively). In addition, ad-iso (R=s-Bu) is expected to exist as a pair of enantiomers. Each of the n-, ad- and co-iso also present as a corresponding cis-/trans-isomeric pair, as well as potentially an enanatiomeric pair derived from the C4 tertiary alcohol adjacent to the ketone to be reduced. In all, not accounting for tautomers of the tetronic acid core, there are up to 16 isomers of iso-α-acids (4 R’s, cis/trans and enantiomer).

Enzymes are typically exquisitely selective for the substrate they act upon. Thus, it is unexpected that a single enzyme or even a simple mixture of two enzymes can completely convert all 16 isomers of iso-α-acids to the corresponding dihydro-(rho)-iso-α-acids. Surprisingly, the present invention comprises a process for converting iso-α-acids to dihydro-(rho)-iso-α-acids using a simple mixture of enzyme(s) and co-factor. Additionally, bio-transformation using enzymes (KREDs) allows for the manufacturing of dihydro-(rho)-iso-α-acids from iso-α-acids to be labelled/certified as “natural”.

The present invention provides engineered ketoreductases capable of indiscriminately reducing the 16 major isomers of iso-α-acids to the corresponding dihydro-(rho)-iso-α-acids by regio-selectively reducing only the ketone on the isoprenyl side chain adjacent to the tertiary alcohol, as depicted in Scheme 1.

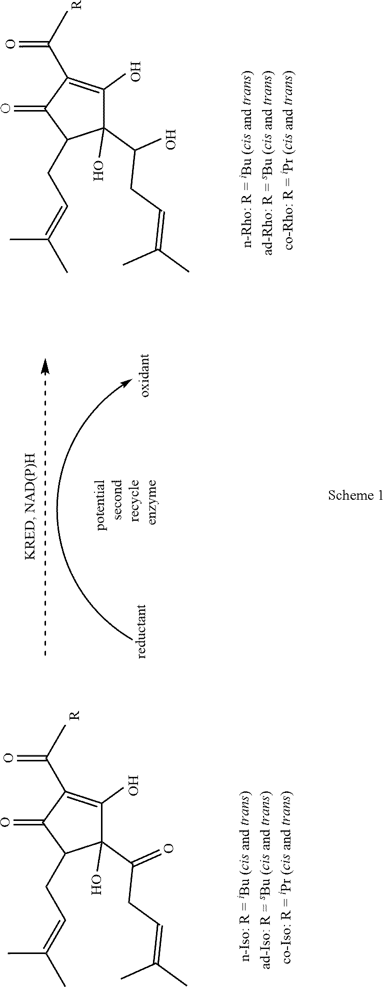

The ketoreductase polypeptide of SEQ ID NO: 4 was selected as the initial backbone for development of the improved enzymes provided by the present invention. The enzyme of SEQ ID NO: 4 is derived from the wild-type ketoreductase from Lactobacillus kefir (SEQ ID NO: 2). The polypeptide of SEQ ID NO: 4 was chosen as the starting backbone due to its high activity in converting iso-α-acids to the corresponding dihydro-(rho)-iso-α-acids, as well as its relative substrate promiscuity and ability to convert a range of isohumulone isomers and epimers to corresponding dihydro-(rho)-iso-α-acid products. Additionally, the polypeptide of SEQ ID NO: 4 displayed activity under a variety of reaction conditions. The wild-type sequence of SEQ ID NO: 2 was found to have no detectable activity in converting iso-α-acids to the corresponding dihydro-(rho)-iso-α-acids.

The engineered ketoreductase polypeptides of the present invention are ketoreductases engineered to have improved properties as compared to the engineered ketoreductase of SEQ ID NO: 4. In some embodiments, the engineered ketoreductase polypeptides of the present invention have improved activity converting iso-α-acids to the corresponding dihydro-(rho)-iso-α-acids as compared to the engineered polypeptide of SEQ ID NO: 4. In some other embodiments, the engineered ketoreductase polypeptides of the present invention have improved activity on a range of iso-α-acid substrates, as compared to the engineered polypeptide of SEQ ID NO: 4. In some other embodiments, the engineered ketoreductase polypeptides of the present invention have improved activity on a range of substrate and cofactor concentrations, as compared to the engineered polypeptide of SEQ ID NO: 4. In some other embodiments, the engineered ketoreductase polypeptides of the present invention have improved activity at high substrate concentrations, as compared to the engineered polypeptide of SEQ ID NO: 4. In some other embodiments, the engineered ketoreductase polypeptides of the present invention have improved activity at low cofactor concentrations, as compared to the engineered polypeptide of SEQ ID NO: 4.

In some embodiments, the engineered ketoreductase polypeptides have improved activity on one or more substrates. In some embodiments, the substrate comprises a mixture of iso-α-acids. In some embodiments, the substrate comprises cis-isohumulone. In some embodiments, the substrate comprises trans-isohumulone. In some embodiments, the substrate comprises cis-isocohumulone. In some embodiments, the substrate comprises trans-isocohumulone. In some embodiments, the substrate comprises cis-isoadhumulone. In some embodiments, the substrate comprises trans-isoadhumulone.

In some embodiments, the engineered ketoreductase polypeptides have improved activity on one or more substrates. In some embodiments, the engineered ketoreductase polypeptides have improved activity on a mixture of iso-α-acids. In some embodiments, the engineered ketoreductase polypeptides have improved activity on cis-isohumulone. In some embodiments, the engineered ketoreductase polypeptides have improved activity on trans-isohumulone. In some embodiments, the engineered ketoreductase polypeptides have improved activity on cis-isocohumulone. In some embodiments, the engineered ketoreductase polypeptides have improved activity on trans-isocohumulone. In some embodiments, the engineered ketoreductase polypeptides have improved activity on cis-isoadhumulone. In some embodiments, the engineered ketoreductase polypeptides have improved activity on trans-isoadhumulone.

In some embodiments, the engineered ketoreductase polypeptides convert substrate compounds to product compounds in the presence of a cofactor recycling system. In some embodiments, the cofactor recycling system compromises a second enzyme, such as glucose dehydrogenase. In some embodiments, the cofactor recycling system comprises isopropanol.

In some embodiments, the engineered ketoreductase polypeptides are capable of converting the substrate compounds to product compounds with an activity that is increased at least about 1.2 fold, 1.5 fold, 2 fold, 3 fold, 4 fold, 5 fold, 10 fold, 20 fold, 30 fold, 40 fold, 50 fold, or 100 fold relative to the activity of the reference polypeptides of SEQ ID NO: 4, 6, 80, 104, 172, 186, 194, 252, 270, 272, 286, 328, and/or 330 under suitable reaction conditions. In some embodiments, the engineered ketoreductase polypeptides are capable of converting the substrate compounds to product compounds with a percent conversion of at least about 40%, at least about 50%, at least about 60%, at least about 70%, at least about 80%, or at least about 90%, at least about 95%, at least about 98%, at least about 99%, in a reaction time of about 48 h, about 36 h, about 24 h, or an even shorter length of time, under suitable reaction conditions.

The suitable reaction conditions can comprise a combination of reaction parameters that provide for the biocatalytic conversion of the substrate compounds to corresponding product compounds. Accordingly, in some embodiments of the process, the combination of reaction parameters comprises: (a) about 0.1 to 220 g/L of substrate compound(s); (b) about 0.5 to 50 g/L engineered polypeptide; (c) about 0.01 to 10 g/L NADP⁺ in about 10-60% isopropanol (d) about 5 to 200 mM triethanolamine∗H₂SO₄; (e) about 0 to 5 mM MgSO₄ or MgCh; (f) temperature of about 25° C. to 60° C.; and (g) pH of 6 to 10. Accordingly, in some embodiments of the process, the combination of reaction parameters comprises: (a) about 10 to 220 g/L of substrate compound(s); (b) about 0.5 to 50 g/L engineered polypeptide; (c) about 0.01 to 10 g/L NADP⁺ in about 10-60% isopropanol (d) about 5 to 200 mM potassium or sodium phosphate; (e) about 0 to 5 mM MgSO₄ or MgCl₂; (f) temperature of about 25° C. to 60° C.; and (g) pH of about 6 to 10.

In some embodiments, the combination of reaction parameters comprises: (a) about 80 g/L of substrate compound ; (b) about 20 g/L engineered polypeptide; (c) about 0.01 g/L NADP⁺ in 40% isopropanol; (d) about 100 mM triethanolamine*H₂SO₄; (e) about 2 mM MgSO₄ ; (f) about 40° C.; and (g) pH of about 8. In some embodiments, the combination of reaction parameters comprises: (a) about 160 g/L of substrate compounds; (b) about 20 g/L engineered polypeptide; (c) about 0.01 g/L NADP⁺ in 40% isopropanol; (d) about 100 mM potassium phosphate; (e) about 2 mM MgSO₄ ; (f) about 40° C.; and (g) pH of about 8.

Further exemplary reaction conditions include the assay conditions provided in the Examples.

In some embodiments, the improved engineered ketoreductase enzymes comprise amino acid residue deletions in the naturally occurring ketoreductase polypeptides or deletions of amino acid residues in other engineered ketoreductase polypeptides. Thus, in some embodiments of the invention, the deletions comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the ketoreductase polypeptides, as long as the functional activity of the ketoreductase is maintained. In some embodiments, the deletions can comprise, 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 amino acid residues. In some embodiments, the number of deletions can be 1, 2, 3, 4, 5, 6, 7, 8, 9, 10, 11, 12, 14, 15, 16, 18, 20, 22, 24, 26, 30, 35 or about 40 amino acids. In some embodiments, the deletions can comprise deletions of 1, 2, 3, 4, 5, 6, 7, 8, 9, 10, 11, 12, 13, 14, 15, 16, 18, or 20 amino acid residues.

As described herein, the ketoreductase polypeptides of the invention can be in the form of fusion polypeptides in which the ketoreductases are fused to other polypeptides, such as antibody tags (e.g., myc epitope) or purifications sequences (e.g., His tags). Thus, in some embodiments, the ketoreductase polypeptides find use with or without fusions to other polypeptides.

In some embodiments, the polypeptides described herein are not restricted to the genetically encoded amino acids. In addition to the genetically encoded amino acids, the polypeptides described herein may be comprised, either in whole or in part, of naturally-occurring and/or synthetic non-encoded amino acids. Certain commonly encountered non-encoded amino acids of which the polypeptides described herein may be comprised include, but are not limited to: the D-stereomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); omithine (Om); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised are apparent to those of skill in the art. These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(δ-benzylester), Gln(xanthyl), Asn(N-8-xanthyl), His(bom), His(benzyl), His(tos), Lys(finoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

As described above the various modifications introduced into the naturally occurring polypeptide to generate an engineered ketoreductase enzyme can be targeted to a specific property of the enzyme.

### Polynucleotides Encoding Engineered Enzymes

In another aspect, the present invention provides polynucleotides encoding the engineered ketoreductase. The polynucleotides may be operatively linked to one or more heterologous regulatory sequences that control gene expression to create a recombinant polynucleotide capable of expressing the polypeptide. Expression constructs containing a heterologous polynucleotide encoding the engineered ketoreductase can be introduced into appropriate host cells to express the corresponding ketoreductase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved ketoreductase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made by selecting combinations based on the possible codon choices, and all such variations are to be considered specifically disclosed for any polypeptide disclosed herein, including the amino acid sequences presented in the Tables in the Examples. In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells.

In some embodiments, the polynucleotide comprises a nucleotide sequence encoding the naturally occurring ketoreductase polypeptide amino acid sequence, as represented by SEQ ID NO: 1. In some embodiments, the polynucleotide has a nucleic acid sequence comprising at least 80%, 85%, 90%, 91%, 92%, 93%, 94%, 95%, 96%, 97%, 98%, 99% or more identity to the nucleic acid sequences of SEQ ID NO: 3, 5, 79, 103, 171, 185, 193, 251, 269, 271, 285, 327 and/or 329 each of which encodes the identical polypeptide sequences of SEQ ID NO: 4, 6, 80, 104, 172, 186, 194, 252, 270, 272, 286, 328, and/or 330, respectively.

In some embodiments, the enzyme polynucleotide encodes an engineered polypeptide having enzyme activity with the properties disclosed herein, wherein the polypeptide comprises an amino acid sequence having at least 60%, 65%, 70%, 75%, 80%, 85%, 86%, 87%, 88%, 89%, 90%, 91%, 92%, 93%, 94%, 95%, 96%, 97%, 98%, 99% or more identity to a reference sequence selected from the SEQ ID NOS provided herein, or the amino acid sequence of any variant (e.g., those provided in the Examples), and one or more residue differences as compared to the reference polynucleotide(s), or the amino acid sequence of any variant as disclosed in the Examples (for example 1, 2, 3, 4, 5, 6, 7, 8, 9, 10 or more amino acid residue positions). In some embodiments, the reference polypeptide sequence is selected from SEQ ID NOS: 4, 6, 80, 104, 172, 186, 194, 252, 270, 272, 286, 328, and/or 330.

In some embodiments, the polynucleotide encoding the engineered ketoreductase comprises a polynucleotide sequence having at least 60%, 65%, 70%, 75%, 80%, 85%, 86%, 87%, 88%, 89%, 90%, 91%, 92%, 93%, 94%, 95%, 96%, 97%, 98%, 99% or more identity to a sequence selected from SEQ ID NOS: 3, 5, 79, 103, 171, 185, 193, 251, 269, 271, 285, 327 and/or 329. In some embodiments, the polynucleotide encoding the engineered ketoreductase comprises SEQ ID NO: 5, 79, 103, 171, 185, 193, 251, 269, 271, 285, 327 and/or 329. In some embodiments, the polynucleotide encoding the engineered ketoreductase comprises a polynucleotide sequence having at least 60%, 65%, 70%, 75%, 80%, 85%, 86%, 87%, 88%, 89%, 90%, 91%, 92%, 93%, 94%, 95%, 96%, 97%, 98%, 99% or more identity to a sequence selected from SEQ ID NOS: 5 to 411. In some embodiments, the polynucleotide encoding the engineered ketoreductase comprises a polynucleotide sequence selected from SEQ ID NOS: 5 to 411.

In some embodiments, the engineered ketoreductase sequences comprise sequences that comprise positions identified to be beneficial, as described in the Examples.

In some embodiments, isolated polynucleotides encoding an improved ketoreductase are manipulated in a variety of ways to provide for improved expression and/or production of the polypeptides. Manipulation of the isolated polynucleotide prior to its insertion into a vector may be desirable or necessary, depending on the expression vector used. The techniques for modifying polynucleotides and nucleic acid sequences utilizing recombinant DNA methods are well known in the art.

For bacterial host cells, suitable promoters for directing transcription of the nucleic acid constructs of the present invention, include the promoters obtained from the E.coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroffet al., Proc. Natl. Acad. Sci. USA 75: 3727-3731 [1978]), as well as the tac promoter (See e.g., DeBoer et al., Proc. Natl Acad. Sci. USA 80: 21-25 [1983]). Additional suitable promoters are known to those in the art.

For filamentous fungal host cells, suitable promoters for directing the transcription of the nucleic acid constructs of the present invention include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters include, but are not limited to those from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, as well as other useful promoters for yeast host cells (See e.g., Romanos et al., Yeast 8:423-488 [1992]).

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase, as well as other useful terminators for yeast host cells known in the art (See e.g,. Romanos et al., supra).

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase., as well as additional useful polyadenylation sequences for yeast host cells known in the art (See e.g., Guo et al., Mol. Cell. Biol., 15:5983-5990 [1995]).

The control sequence may also be a signal peptide coding region that codes for an amino acid sequence linked to the amino terminus of a polypeptide and directs the encoded polypeptide into the cell’s secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NClB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA,, as well as additional signal peptides known in the art (See e.g., Simonen et al., Microbiol. Rev., 57: 109-137 [1993]).

Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells can be from the genes for Saccharomyces cerevisiae alpha-factor and Saccharomyces cerevisiae invertase, as well as additional useful signal peptide coding regions (See e.g., Romanos et al., 1992, supra).

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the KRED polypeptide of the present invention would be operably linked with the regulatory sequence.

Thus, in some embodiments, the present invention is also directed to a recombinant expression vector comprising a polynucleotide encoding an engineered ketoreductase polypeptide or a variant thereof, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector (i.e., a vector that exists as an extrachromosomal entity), the replication of which is independent of chromosomal replication, (e.g., a plasmid, an extrachromosomal element, a minichromosome, or an artificial chromosome). The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker can be a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present invention can contain an element(s) that permits integration of the vector into the host cell’s genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori or the origins of replication of plasmids pBR322, pUC19, pACYC177 (which plasmid has the P15A ori), or pACYC184 permitting replication in E. coli, and pUB110, pE194, pTA1060, or pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433 [1978]).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

It is not intended that the present invention be limited to the expression vectors disclosed herein. Those of skill in the art will recognize that any suitable expression vector may be used in the present invention. Many of the expression vectors for use in the present invention are commercially available. Suitable commercial expression vectors include, but are not limited to p3xFLAGTM™ expression vectors (Sigma-Aldrich), which include a CMV promoter and hGH polyadenylation site for expression in mammalian host cells and a pBR322 origin of replication and ampicillin resistance markers for amplification in E. coli. Other commercially available suitable expression vectors include but are not limited to the pBluescriptII SK(-) and pBK-CMV vectors (Stratagene), and plasmids derived from pBR322 (Gibco BRL), pUC (Gibco BRL), pREP4, pCEP4 (Invitrogen) or pPoly (See, Lathe et al., Gene 57:193-201 [1987]).

### Host Cells for Expression of Engineered Polypeptides

The present invention also provides a host cell comprising a polynucleotide encoding an improved ketoreductase polypeptide of the present invention, the polynucleotide being operatively linked to one or more control sequences for expression of the ketoreductase enzyme in the host cell. Host cells for use in expressing the KRED polypeptides encoded by the expression vectors of the present invention are well known in the art and include but are not limited to, bacterial cells, such as E. coli, Lactobacillus kefir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture media and growth conditions for the above-described host cells are well known in the art.

Polynucleotides for expression of the ketoreductase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

Escherichia coli W3110 is a host strain that finds use in the present invention, although it is not intended that the present invention be limited to this specific host strain. The expression vector was created by operatively linking a polynucleotide encoding an improved enzyme into the plasmid pCK1 10900 operatively linked to the lac promoter under control of the lacI repressor. The expression vector also contained the P15a origin of replication and the chloramphenicol resistance gene. Cells containing the subject polynucleotide in Escherichia coli W3110 can be isolated by subjecting the cells to chloramphenicol selection.

### Methods of Generating Engineered Ketoreductase Polypeptides

In some embodiments, to make the improved KRED polynucleotides and polypeptides of the present invention, the naturally-occurring ketoreductase enzyme that catalyzes the reduction reaction is obtained (or derived) from Lactobacillus kefir. In some embodiments, the parent polynucleotide sequence is codon optimized to enhance expression of the ketoreductase in a specified host cell. As an illustration, the parental polynucleotide sequence encoding the wild-type KRED polypeptide of Lactobacillus kefir was constructed from oligonucleotides prepared based upon the known polypeptide sequence of Lactobacillus kefir KRED sequence available from the Genbank database. The parental polynucleotide sequence was codon optimized for expression in E. coli and the codon-optimized polynucleotide cloned into an expression vector, placing the expression of the ketoreductase gene under the control of the lac promoter and lacI repressor gene. Clones expressing the active ketoreductase in E. coli were identified and the genes sequenced to confirm their identity.

In some embodiments, the engineered ketoreductases are obtained by subjecting the polynucleotide encoding the naturally occurring ketoreductase to mutagenesis and/or directed evolution methods, as discussed above. Mutagenesis may be performed in accordance with any of the techniques known in the art, including random and site-specific mutagenesis. Directed evolution can be performed with any of the techniques known in the art to screen for improved promoter variants including shuffling. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related non-US counterparts; Ling et al., Anal. Biochem., 254(2):157-78 [1997]; Dale etal., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer etal., Cell, 38:879-887 [1984]; Wells etal., Gene, 34:315-323 [1985]; Minshull et al., Curr. Op. Chem. Biol., 3: 284-290 [1999]; Christians et al., Nat. Biotechnol., 17: 259-264 [1999]; Crameri et al., Nature, 391: 288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

The clones obtained following mutagenesis treatment are screened for engineered ketoreductases having a desired improved enzyme property. Measuring enzyme activity from the expression libraries can be performed using the standard biochemistry technique of monitoring the rate of decrease (via a decrease in absorbance or fluorescence) of NADH or NADPH concentration, as it is converted into NAD⁺ or NADP⁺. In this reaction, the NADH or NADPH is consumed (oxidized) by the ketoreductase as the ketoreductase reduces a ketone substrate to the corresponding hydroxyl group. The rate of decrease of NADH or NADPH concentration, as measured by the decrease in absorbance or fluorescence, per unit time indicates the relative (enzymatic) activity of the KRED polypeptide in a fixed amount of the lysate (or a lyophilized powder made therefrom). The stereochemistry of the products can be ascertained by various known techniques, and as provided in the Examples. Where the improved enzyme property desired is thermal stability, enzyme activity may be measured after subjecting the enzyme preparations to a defined temperature and measuring the amount of enzyme activity remaining after heat treatments. Clones containing a polynucleotide encoding a ketoreductase are then isolated, sequenced to identify the nucleotide sequence changes (if any), and used to express the enzyme in a host cell.

Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides of the invention can be prepared by chemical synthesis (e.g., using the classical phosphoramidite method described by Beaucage et al., Tet. Lett., 22: 1859-69 [1981], or the method described by Matthes et al., EMBO J., 3:801-05 [1984], as it is typically practiced in automated synthetic methods). According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources (e.g., The Midland Certified Reagent Company, Midland, TX, The Great American Gene Company, Ramona, CA, ExpressGen Inc. Chicago, IL, Operon Technologies Inc., Alameda, CA, and many others).

Engineered ketoreductase enzymes expressed in a host cell can be recovered from the cells and or the culture medium using any one or more of the well known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ (Sigma-Aldrich).

Chromatographic techniques for isolation of the ketoreductase polypeptides include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques are used to isolate the improved ketoreductase enzymes. For affinity chromatography purification, any antibody which specifically binds the ketoreductase polypeptide may be used. For the production of antibodies, various host animals, including but not limited to rabbits, mice, rats, etc., may be immunized by injection with the ketoreductase. The ketoreductase polypeptide may be attached to a suitable carrier, such as BSA, by means of a side chain functional group or linkers attached to a side chain functional group. Various adjuvants may be used to increase the immunological response, depending on the host species, including but not limited to Freund’s (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacillus Calmette Guerin) and Corynebacterium parvum.

The ketoreductases may be prepared and used in the form of cells expressing the enzymes, as crude extracts, or as isolated or purified preparations. The ketoreductases may be prepared as lyophilizates, in powder form (e.g., acetone powders), or prepared as enzyme solutions. In some embodiments, the ketoreductases can be in the form of substantially pure preparations.

In some embodiments, the ketoreductase polypeptides can be attached to a solid substrate. The substrate can be a solid phase, surface, and/or membrane. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of the substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

As is known by those of skill in the art, ketoreductase-catalyzed reduction reactions typically require a cofactor. Reduction reactions catalyzed by the engineered ketoreductase enzymes described herein also typically require a cofactor, although many embodiments of the engineered ketoreductases require far less cofactor than reactions catalyzed with wild-type ketoreductase enzymes. As used herein, the term “cofactor” refers to a non-protein compound that operates in combination with a ketoreductase enzyme. Cofactors suitable for use with the engineered ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺). Generally, the reduced form of the cofactor is added to the reaction mixture. The reduced NAD(P)H form can be optionally regenerated from the oxidized NAD(P)⁺ form using a cofactor regeneration system. The term “cofactor regeneration system” refers to a set of reactants that participate in a reaction that reduces the oxidized form of the cofactor (e.g., NADP⁺ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. The cofactor regeneration system may also comprise a cosubstrate such as isopropanol. Cofactor regeneration systems to regenerate NADH or NADPH from NAD⁺ or NADP⁺, respectively, are known in the art and may be used in the methods described herein.

### Experimental

Various features and embodiments of the invention are illustrated in the following representative examples, which are intended to be illustrative, and not limiting.

In the experimental disclosure below, the following abbreviations apply: ppm (parts per million); M (molar); mM (millimolar), uM and µM (micromolar); nM (nanomolar); mol (moles); gm and g (gram); mg (milligrams); ug and µg (micrograms); L and 1 (liter); ml and mL (milliliter); cm (centimeters); mm (millimeters); µm and µm (micrometers); sec. (seconds); min(s) (minute(s)); h(s) and hr(s) (hour(s)); U (units); MW (molecular weight); rpm (rotations per minute); °C (degrees Centigrade); RT (room temperature); CDS (coding sequence); DNA (deoxyribonucleic acid); RNA (ribonucleic acid); HPLC (high performance liquid chromatography); FIOPC (fold improvement over positive control); HTP (high throughput); LB (Luria broth); KPO₄ (potassium phosphate); KPO₃ (potassium phosphite); TEoA (triethanolamine); PMBS (polymyxin B sulfate); Sigma-Aldrich (Sigma-Aldrich, St. Louis, MO); Millipore (Millipore, Corp., Billerica MA); Difco (Difco Laboratories, BD Diagnostic Systems, Detroit, MI); Daicel (Daicel, West Chester, PA); Genetix (Genetix USA, Inc., Beaverton, OR); Molecular Devices (Molecular Devices, LLC, Sunnyvale, CA); Applied Biosystems (Applied Biosystems, part of Life Technologies, Corp., Grand Island, NY), Agilent (Agilent Technologies, Inc., Santa Clara, CA); Thermo Scientific (part of Thermo Fisher Scientific, Waltham, MA); Corning (Corning, Inc., Palo Alto, CA); and Bio-Rad (Bio-RadLaboratories, Hercules, CA).

The following sequences were used in the development of the present invention.

SEQ ID NO: 413:

aacggctttgccgcggccctctcacttccctgttaagtatcttcctggca
tcttccaggaaatctccgccccgttcgtaagccatttccgctcgccgcag
tcgaacgaccgagcgtagcgagtcagtgagcgaggaagcggaatatatcc
tgtatcacatattctgctgacgcaccggtgcagccttttttctcctgcca
catgaagcacttcactgacaccctcatcagtgaaccaccgctggtagcgg
tggtttttttaggcctatggccttttttttttgtgggaaacctttcgcgg
tatggtattaaagcgcccggaagagagtcaattcagggtggtgaatgtga
aaccagtaacgttatacgatgtcgcagagtatgccggtgtctcttatcag
accgtttcccgcgtggtgaaccaggccagccacgtttctgcgaaaacgcg
ggaaaaagtggaagcggcgatggcggagctgaattacattcccaaccgcg
tggcacaacaactggcgggcaaacagtcgttgctgattggcgttgccacc
tccagtctggccctgcacgcgccgtcgcaaattgtcgcggcgattaaatc
tcgcgccgatcaactgggtgccagcgtggtggtgtcgatggtagaacgaa
gcggcgtcgaagcctgtaaagcggcggtgcacaatcttctcgcgcaacgc
gtcagtgggctgatcattaactatccgctggatgaccaggatgccattgc
tgtggaagctgcctgcactaatgttccggcgttatttcttgatgtctctg
accagacacccatcaacagtattattttctcccatgaagacggtacgcga
ctgggcgtggagcatctggtcgcattgggtcaccagcaaatcgcgctgtt
agcgggcccattaagttctgtctcggcgcgtctgcgtctggctggctggc
ataaatatctcactcgcaatcaaattcagccgatagcggaacgggaaggc
gactggagtgccatgtccggttttcaacaaaccatgcaaatgctgaatga
gggcatcgtttccactgcgatgctggttgccaacgatcagatggcgctgg
gcgcaatgcgcgccattaccgagtccgggctgcgcgttggtgcggacatc
tcggtagtgggatacgacgataccgaagacagctcatgttatatcccgcc
gttaaccaccatcaaacaggattttcgcctgctggggcaaaccagcgtgg
accgcttgctgcaactctctcagggccaggcggttaagggcaatcagctg
ttgcccgtctcactggtgaaaagaaaaaccaccctggcgcccaatacgca
aaccgcctctccccgcgcgttggccgattcattaatgcagctggcacgac
aggtttcccgactggaaagcgggcagtgagcggtacccgataaaagcggc
ttcctgacaggaggccgttttgtttctcgagttaattaaggcagtgagcg
caacgcaattaatgtgagttagctcactcattaggcaccccaggctttac
actttatgcttccggctcgtatgttgtgtggaattgtgagcggataacaa
tttcacacaggaaacggctatgaccatgattacggattcactggccgtcg
ttttacaatctagaggccagcctggccataaggagatatacatatgagta
ttcaacatttccgtgtcgcccttattcccttttctgcggcattttgcctt
cctgtttttgctcacccagaaacgctggtgaaagtaaaagatgctgaaga
tcagttgggtgcacgagtgggttacatcgaactggatctcaacagcggta
agatccttgagagttttcgccccgaagagcgttttccaatgatgagcact
tttaaagttctgctatgtggcgcggtattatcccgtgttgacgccgggca
agagcaactcggtcgccgcatacactattctcagaatgacttggttgagt
actcaccagtcacagaaaagcatcttacggatggcatgacagtaagagaa
ttatgcagtgctgccataaccatgagtgataacactgcggccaacttact
tctgacaacgatcggaggaccgaaggagctaaccgtttttttgcacacca
tgggggatcatgtaactcgccttgatcgttgggaaccggagctgaatgaa
gccataccaaacgacgagcgtgacaccacgatgcctacagcaatggcaac
aacgttgcgcaaactattaactggcgaactacttactctagcttcccggc
aacaattaatagactggatggaggcggataaagttgcaggaccacttctg
cgctcggcccttccggctggctggtttattgctgataaatctggagccgg
tgagcgtgggtctcgcggtatcattgcagcactggggccagatggtaagc
cctcccgtatcgtagttatctacacgacggggagtcaggcaactatggat
gaacgtaatagacagatcgctgagataggtgcctcactgattaagcattg
gggccaaactggccaccatcaccatcaccattagggaagagcagatgggc
aagcttgacctgtgaagtgaaaaatggcgcacattgtgcgacattttttt
ttgaattctacgtaaaaagcagccgatacatcggctgctttttttttctg
cagggtgaaacaaaacggttgaccacatgaagtaaacacggtacggtgcg
gtagtttatcacagttaaattgctaacgcagtcaggcaaagtccatgggt
tttctttccggtaagcgcattctggtaaccggtgttgccagcaaactatc
catcgcctacggtatcgctcaggcgatgcaccgcgaaggagctgaactgg
cattcacctaccagaacgacaaactgaaaggccgcgtagaagaatttgcc
gctcaattgggttctgacatcgttctgcagtgcgatgttgcagaagatgc
cagcatcgacaccatgttcgctgaactggggaaagtttggccgaaatttg
acggttttgtacactctattgtttttgcacctggcgatcagctggatggt
gactatgttaacgccgttacccgtgaaggcttcaaaattgcccacgacat
cagctcctacagcttcgttgcaatggcaaaagcttgccgctccatgctga
atccgggttctgccctgctgaccctttcctaccttggcgctgagcgcgct
atcccgaactacaacgttatgggtctggcaaaagcgtctctggaagcgaa
cgtgcgctatatggcgaacgcgatgggtccggaaggtgtgcgtgttaacg
ccatctctgctggtccgatccgtactctggcggcctccggtatcaaagac
ttccgcaaaatgctggctcattgcgaagccgttaccccgattcgccgtac
cgttactattgaagatgtgggtaactctgcggcattcctgtgctccgatc
tctctgccggtatctccggtgaagtggtccacgttgacggcggtttcagc
attgctgcaatgaacgaactcgaactgaaataactgcaggagctcaaaca
gcagcctgtattcaggctgctttttagaaatattttatctgattaataag
atgatcttcttgagatcgttttggtctgcgcgtattctcttgctctgaaa
acgaaaaaaccgccttgcagggcggtttttcgaaggttctctgagctacc
aactctttgaaccgaggtaactggcttggaggagcgcagtcaccaaaact
tgtcctttcagtttagccttaaccggcgcatgacttcaagactaactcct
ctaaatcaattaccagtggctgctgccagtggtgcttttgcatgtctttc
cgggttggactcaagacgatagttaccggataaggcgcagcggtcggact
gaacggggggttcgtgcatacagtccagcttggagcgaactgcctacccg
gaactgagtgtcaggcgtggaatgagacaaacgcggccataacagcggaa
tgacaccggtaaaccgaaaggcaggaacaggagagcgcacgagggagccg
ccagggggaaacgcctggtatctttatagtcctgtcgggtttcgccacca
ctgatttgagcgtcagatttcgtgatgcttgtcaggggggcggagcctat
ggaaa (SEQ ID NO: 413)

SEQ ID NO: 414:

aacggctttgccgcggccctctcacttccctgttaagtatcttcctggca
tcttccaggaaatctccgccccgttcgtaagccatttccgctcgccgcag
tcgaacgaccgagcgtagcgagtcagtgagcgaggaagcggaatatatcc
tgtatcacatattctgctgacgcaccggtgcagccttttttctcctgcca
catgaagcacttcactgacaccctcatcagtgaaccaccgctggtagcgg
tggtttttttaggcctatggccttttttttttgtgggaaacctttcgcgg
tatggtattaaagcgcccggaagagagtcaattcagggtggtgaatgtga
aaccagtaacgttatacgatgtcgcagagtatgccggtgtctcttatcag
accgtttcccgcgtggtgaaccaggccagccacgtttctgcgaaaacgcg
ggaaaaagtggaagcggcgatggcggagctgaattacattcccaaccgcg
tggcacaacaactggcgggcaaacagtcgttgctgattggcgttgccacc
tccagtctggccctgcacgcgccgtcgcaaattgtcgcggcgattaaatc
tcgcgccgatcaactgggtgccagcgtggtggtgtcgatggtagaacgaa
gcggcgtcgaagcctgtaaagcggcggtgcacaatcttctcgcgcaacgc
gtcagtgggctgatcattaactatccgctggatgaccaggatgccattgc
tgtggaagctgcctgcactaatgttccggcgttatttcttgatgtctctg
accagacacccatcaacagtattattttctcccatgaagacggtacgcga
ctgggcgtggagcatctggtcgcattgggtcaccagcaaatcgcgctgtt
agcgggcccattaagttctgtctcggcgcgtctgcgtctggctggctggc
ataaatatctcactcgcaatcaaattcagccgatagcggaacgggaaggc
gactggagtgccatgtccggttttcaacaaaccatgcaaatgctgaatga
gggcatcgtttccactgcgatgctggttgccaacgatcagatggcgctgg
gcgcaatgcgcgccattaccgagtccgggctgcgcgttggtgcggacatc
tcggtagtgggatacgacgataccgaagacagctcatgttatatcccgcc
gttaaccaccatcaaacaggattttcgcctgctggggcaaaccagcgtgg
accgcttgctgcaactctctcagggccaggcggttaagggcaatcagctg
ttgcccgtctcactggtgaaaagaaaaaccaccctggcgcccaatacgca
aaccgcctctccccgcgcgttggccgattcattaatgcagctggcacgac
aggtttcccgactggaaagcgggcagtgagcggtacccgataaaagcggc
ttcctgacaggaggccgttttgtttctcgagttaattaaggcagtgagcg
caacgcaattaatgtgagttagctcactcattaggcaccccaggctttac
actttatgcttccggctcgtatgttgtgtggaattgtgagcggataacaa
tttcacacaggaaacggctatgaccatgattacggattcactggccgtcg
ttttacaatctagaggccagcctggccataaggagatatacatatgagta
ttcaacatttccgtgtcgcccttattcccttttctgcggcattttgcctt
cctgtttttgctcacccagaaacgctggtgaaagtaaaagatgctgaaga
tcagttgggtgcacgagtgggttacatcgaactggatctcaacagcggta
agatccttgagagttttcgccccgaagagcgttttccaatgatgagcact
tttaaagttctgctatgtggcgcggtattatcccgtgttgacgccgggca
agagcaactcggtcgccgcatacactattctcagaatgacttggttgagt
actcaccagtcacagaaaagcatcttacggatggcatgacagtaagagaa
ttatgcagtgctgccataaccatgagtgataacactgcggccaacttact
tctgacaacgatcggaggaccgaaggagctaaccgtttttttgcacacca
tgggggatcatgtaactcgccttgatcgttgggaaccggagctgaatgaa
gccataccaaacgacgagcgtgacaccacgatgcctacagcaatggcaac
aacgttgcgcaaactattaactggcgaactacttactctagcttcccggc
aacaattaatagactggatggaggcggataaagttgcaggaccacttctg
cgctcggcccttccggctggctggtttattgctgataaatctggagccgg
tgagcgtgggtctcgcggtatcattgcagcactggggccagatggtaagc
cctcccgtatcgtagttatctacacgacggggagtcaggcaactatggat
gaacgtaatagacagatcgctgagataggtgcctcactgattaagcattg
gggccaaactggccaccatcaccatcaccattagggaagagcagatgggc
aagcttgacctgtgaagtgaaaaatggcgcacattgtgcgacattttttt
ttgaattctacgtaaaaagcagccgatacatcggctgctttttttttctg
cagggtgaaacaaaacggttgactacatgaagtaaacacggtacggtgcg
gtagtttatcacagttaaattgctaacgcagtcaggcaaagtccatgggt
tttctttccggtaagcgcattctggtaaccggtgttgccagcaaactatc
catcgcctacggtatcgctcaggcgatgcaccgcgaaggagctgaactgg
cattcacctaccagaacgacaaactgaaaggccgcgtagaagaatttgcc
gctcaattgggttctgacatcgttctgcagtgcgatgttgcagaagatgc
cagcatcgacaccatgttcgctgaactggggaaagtttggccgaaatttg
acggttttgtacactctattgtttttgcacctggcgatcagctggatggt
gactatgttaacgccgttacccgtgaaggcttcaaaattgcccacgacat
cagctcctacagcttcgttgcaatggcaaaagcttgccgctccatgctga
atccgggttctgccctgctgaccctttcctaccttggcgctgagcgcgct
atcccgaactacaacgttatgggtctggcaaaagcgtctctggaagcgaa
cgtgcgctatatggcgaacgcgatgggtccggaaggtgtgcgtgttaacg
ccatctctgctggtccgatccgtactctggcggcctccggtatcaaagac
ttccgcaaaatgctggctcattgcgaagccgttaccccgattcgccgtac
cgttactattgaagatgtgggtaactctgcggcattcctgtgctccgatc
tctctgccggtatctccggtgaagtggtccacgttgacggcggtttcagc
attgctgcaatgaacgaactcgaactgaaataactgcaggagctcaaaca
gcagcctgtattcaggctgctttttagaaatattttatctgattaataag
atgatcttcttgagatcgttttggtctgcgcgtaatctcttgctctgaaa
acgaaaaaaccgccttgcagggcggtttttcgaaggttctctgagctacc
aactctttgaaccgaggtaactggcttggaggagcgcagtcaccaaaact
tgtcctttcagtttagccttaaccggcgcatgacttcaagactaactcct
ctaaatcaattaccagtggctgctgccagtggtgcttttgcatgtctttc
cgggttggactcaagacgatagttaccggataaggcgcagcggtcggact
gaacggggggttcgtgcatacagtccagcttggagcgaactgcctacccg
gaactgagtgtcaggcgtggaatgagacaaacgcggccataacagcggaa
tgacaccggtaaaccgaaaggcaggaacaggagagcgcacgagggagccg
ccagggggaaacgcctggtatctttatagtcctgtcgggtttcgccacca
ctgatttgagcgtcagatttcgtgatgcttgtcaggggggcggagcctat
ggaaa (SEQ ID NO: 414)

### Example 1

### E. Coli Expression Hosts Containing Recombinant KRED Genes

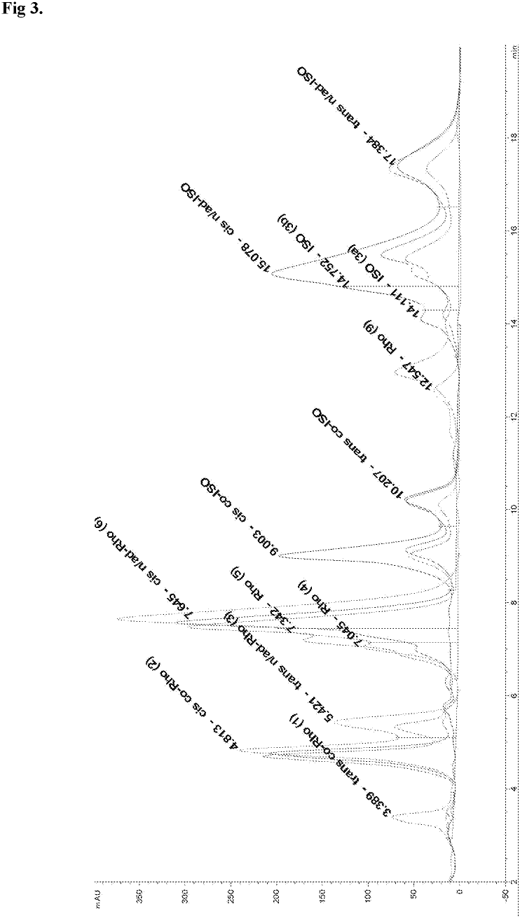

The initial KRED enzymes used to produce the variants of the present invention were obtained from Codexis’s collection of commercially available KRED enzyme panels. During the initial screen, the variant of SEQ ID NO: 4 produced the most product as determined by LC/MS. The KRED-encoding genes were cloned into an expression vector system, including pCK110900 (See, FIG. 3 of U.S. Pat. Appln. Publn. No. 2006/0195947), SEQ ID NO: 413, or SEQ ID NO: 414, operatively linked to the lac promoter under control of the lacl repressor. The expression vector system also contains the P15a origin of replication and a chloramphenicol resistance gene. It is not intended that the present invention be limited to the expression vectors disclosed herein. Those of skill in the art will recognize that any suitable expression vector may be used in the present invention, including, but not limited to p3xFLAGTM™ expression vectors (Sigma-Aldrich), the pBluescriptII SK(-) and pBK-CMV vectors (Stratagene), and plasmids derived from pBR322 (Gibco BRL), pUC (Gibco BRL), pREP4, pCEP4 (Invitrogen) or pPoly (See, Lathe et al., Gene 57:193-201 [1987]).

The resulting plasmids were transformed into E. coli W3110, using standard methods known in the art. The transformants were isolated by subjecting the cells to chloramphenicol selection, as known in the art (See e.g., U.S. Pat. No. 8,383,346 and WO2010/144103).

### Example 2

### Preparation of HTP KRED-Containing Wet Cell Pellets

E. coli cells containing recombinant KRED-encoding genes from monoclonal colonies were inoculated into 190 µl LB containing 1% glucose and 30 µg/mL chloramphenicol in the wells of 96-well shallow-well microtiter plates. The plates were sealed with O2-permeable seals, and cultures were grown overnight at 20° C., 200 rpm, and 85% humidity. Then, 20 µl of each of the cell cultures were transferred into the wells of 96-well deep-well plates containing 380 µL TB and 30 µg/mL CAM. The deep-well plates were sealed with O2-permeable seals and incubated at 30° C., 250 rpm, and 85% humidity until an OD600 of 0.6-0.8 was reached. The cell cultures were then induced by IPTG to a final concentration of 1 mM and incubated overnight under the same conditions as originally used. The cells were then pelleted using centrifugation at 4° C., 4000 rpm for 10 min. The supernatants were discarded, and the pellets frozen at -80° C. prior to lysis.

### Example 3

### Preparation of HTP KRED-Containing Cell Lysates

First, the cell pellets that were produced as described in Example 2 were lysed by adding 150 µL lysis buffer containing 100 mM pH 8 triethanolamine∗H₂SO₄ with 2 mM MgSO₄ or 100 mM pH 8 Potassium Phosphate with 2 mM MgSO₄, 1 g/L lysozyme, and 0.5 g/L PMBS. Then, the cell pellets were shaken at room temperature for 2 hours on a bench top shaker. The plates were centrifuged at 4000 rpm, for 15 minutes at 4° C. to remove cell debris. The supernatants were then used in biocatalytic reactions to determine their activity levels.

### Example 4

### Preparation of Lyophilized Lysates From Shake Flask (SF) Cultures

Shake-flask procedures can be used to generate engineered KRED polypeptide shake-flask powders (SFP), which are useful for secondary screening assays and/or use in the biocatalytic processes described herein. Shake flask powder (SFP) preparation of enzymes provides a more purified preparation (e.g., up to 30% of total protein) of the engineered enzyme, as compared to the cell lysate used in HTP assays and also allows for the use of more concentrated enzyme solutions. To start this, selected HTP cultures grown as described above were plated onto LB agar plates with 1% glucose and 30 µg/ml chloramphenicol (CAM), and grown overnight at 37° C. A single colony from each culture was transferred to 6 ml of LB with 1% glucose and 30 µg/ml CAM. The cultures were grown for 18 h at 30° C. at 250 rpm, and subcultured approximately 1:50 into 250 ml of TB containing 30 µg/ml CAM, to a final OD₆₀₀ of 0.05. The cultures were grown for approximately 3 hours at 30° C. at 250 rpm to an OD₆₀₀ between 0.8-1.0 and induced with 1 mM IPTG. The cultures were then grown for 20 h at 30° C. at 250 rpm. The cultures were centrifuged (4000 rpm for 20 min at 4° C.). The supernatant was discarded, and the pellets were re-suspended in 35 ml of 50 mM pH 8 Potassium Phosphate with 2 mM MgSO₄. The re-suspended cells were centrifuged (4000 rpm for 20 min at 4° C.). The supernatant was discarded, and the pellets were re-suspended in 6 ml of 50 mM pH 8 Potassium Phosphate with 2 mM MgSO₄, and the cells were lysed using a cell disruptor from Constant Systems (One Shot). The lysates were pelleted (10,000 rpm for 60 min at 4° C.), and the supernatants were frozen and lyophilized to generate shake flake (SF) enzymes.

### Example 5

### Evolution and Screening of Engineered Polypeptides Derived From SEQ ID NO: 4 for Improved KRED Activity

SEQ ID NO: 4 was selected as the parent enzyme based on the results of screening variants for the reduction of the iso-α-acid substrate. Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 4 (i.e., SEQ ID NO: 3), was used to generate the further engineered polypeptides of Table 5-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 4 using directed evolution methods as described above together with the HTP assay and analytical methods described below in Table 5-2.

Directed evolution began with the polynucleotide set forth in SEQ ID NO: 3. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-well format, in 200 µL total volume/well, which included 50% v/v HTP enzyme lysate, 8 g/L iso-α-acid substrate (Isolone® Isomerized Hop Extract Solution, Kalsec), and 0.1 g/L NADP in 40 vol% isopropanol (IPA) in 100 mM pH 8 triethanolamine∗H₂SO₄ with 2 mM MgSO₄. The plates were sealed and incubated at 40° C. with shaking at 600 rpm for 20-24 hours.

After 20-24 hours, 1000 µL of acetonitrile with 0.1% acetic acid was added. The plates were sealed and centrifuged at 4000 rpm at 4° C. for 10 min. The quenched sample was further diluted 4-5x in 50:50 acetonitrile:water mixture prior to HPLC analysis. The HPLC run parameters are described below in Table 5-2.

TABLE 5-1

KRED Variant Activity Relative to SEQ ID NO: 4

| SEQ ID NO: (nt/aa) | Amino Acid Differences (Relative to SEQ ID NO: 4) | Percent Conversion Fold Improvement (Relative to SEQ ID NO: 4)¹ |
|---|---|---|
| 5/6 | L196K | ++++ |
| ⅞ | L196R | +++ |
| 9/10 | L153R | +++ |
| 11/12 | L145S | +++ |
| 13/14 | T152G | ++ |
| 15/16 | L196H | ++ |
| 17/18 | I93V | ++ |
| 19/20 | L145M | ++ |
| 21/22 | L145G | ++ |
| 23/24 | L145C | + |
| 25/26 | L153V | + |
| 27/28 | D197R | + |
| 29/30 | L21R | + |
| 31/32 | I93D | + |
| 33/34 | V148I | + |
| 35/36 | L153C | + |
| 37/38 | E200Q | + |
| 39/40 | P194R | + |
| 41/42 | E200R | + |
| 43/44 | M206V | + |
| 45/46 | I93M | + |
| 47/48 | D197G | + |
| 49/50 | P194N | + |
| 51/52 | E200L | + |
| 53/54 | L110I | + |
| 55/56 | I226L | + |
| 57/58 | I93T | + |
| 59/60 | T212S | + |
| 61/62 | P194H | + |
| 63/64 | T152S | + |
| 65/66 | V12I | + |
| 67/68 | K97G | + |
| 69/70 | V87L | + |

¹Levels of increased activity were determined relative to the reference polypeptide of SEQ ID NO: 4 and defined as follows: “+” >1.0 but < 2.0, “++” ≥2 but ≤ 4, “+++” ≥ 4 but ≤ 8, “++++” ≥ 8

TABLE 5-2

HPLC Parameters

| Instrument | Agilent 1100 HPLC | | |
|---|---|---|---|
| Column | 30 × 50 mm 2.7 µm Waters XBridge Phenyl column | | |
| Mobile Phase | A: 0.1% acetic acid in water, B: 0.1% acetic acid in acetonitrile | | |
| Run parameters | 42:58 A/B for 1 minute; ramp to 10:90 A/B over 1 minute | | |
| Flow Rate | 1.5 mL/min | | |
| Run time | 2.0 min | | |
| | Compound | retention time [min] | note |
| Peak Retention Times | Iso-1 | 0.6 | mixture of co-Iso isomers |
| | Iso-2 | 0.7 | mixture of n/ad-Iso isomers |
| | Iso-3 | 0.8 | mixture of n/ad-Iso isomers |
| | Rho-1 | 1.0 | mixture of co-Rho isomers |
| | Rho-2 | 1.2 | mixture of n/ad-Rho isomers |
| | Rho-3 | 1.4 | mixture of n/ad-Rho isomers |
| Column Temperature | 50° C. | | |
| Injection Volume | 10 µL | | |
| Detection | 260 nm | | |

### Example 6

### Evolution and Screening of Engineered Polypeptides Derived From SEQ ID NO: 6 for Improved KRED Activity

Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 6 (i.e., SEQ ID NO: 5), was used to generate the further engineered polypeptides of Table 6-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 6 using directed evolution methods as described above together with the HTP assay and analytical methods described below in Table 5-2.

Directed evolution began with the polynucleotide set forth in SEQ ID NO: 5. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-well format, in 200 µL total volume/well, which included 50% v/v HTP enzyme lysate, 16 or 40 g/L of iso-α-acid substrate (Isolone® Isomerized Hop Extract Solution, Kalsec), and 0.1 g/L NADP in 40 vol% isopropanol (IPA) in 100 mM pH 8 triethanolamine∗H₂SO₄ with 2 mM MgSO₄. The plates were sealed and incubated at 40° C. with shaking at 600 rpm for 20-24 hours.

After 20-24 hours, 1000 µL of acetonitrile with 0.1% acetic acid was added. The plates were sealed and centrifuged at 4000 rpm at 4° C. for 10 min. The quenched sample was further diluted 10-20x in 50:50 acetonitrile:water mixture prior to HPLC analysis. The HPLC run parameters are described in Table 5-2.

TABLE 6-1

KRED Variant Activity Relative to SEQ ID NO: 6

| SEQ ID NO: (nt/aa) | Amino Acid Differences (Relative to SEQ ID NO: 6) | Percent Conversion Fold Improvement (Relative to SEQ ID NO: 6)¹ |
|---|---|---|
| 71/72 | V12I;L145M | ++++ |
| 73/74 | L145M | +++ |
| 75/76 | V87L;L110I;L145M | +++ |
| 77/78 | L145M;T152G | +++ |
| 79/80 | V87L;L110I;L145M | +++ |
| 81/82 | V12I;L110I;L145M;T152G | ++ |
| 83/84 | L110I;L145M;P194H | ++ |
| 85/86 | L110I;L145M;T152G;D197G | + |
| 87/88 | V87L;L110I;L145M;P194N | + |
| 89/90 | V87L;L110I;L145M;P194H | + |
| 91/92 | T152S | + |
| 93/94 | L145M;D197G;I226L | + |
| 95/96 | V87L;L145M;P194H | + |
| 97/98 | L110I | + |

¹Levels of increased activity were determined relative to the reference polypeptide of SEQ ID NO: 6 and defined as follows: “+” >1.0 but < 2.0, “++” ≥2 but ≤ 4, “+++” ≥ 4 but ≤ 8, “++++” ≥ 8

### Example 7

### Evolution and Screening of Engineered Polypeptides Derived From SEQ ID NO: 80 for Improved KRED Activity

Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 80 (i.e., SEQ ID NO: 79), was used to generate the further engineered polypeptides of Table 7-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 80 using directed evolution methods as described above, together with the HTP assay and analytical methods described below in Table 5-2.

Directed evolution began with the polynucleotide set forth in SEQ ID NO: 79. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-well format, in 200 µL total volume/well, which included 25% v/v HTP enzyme lysate, 60 or 80 g/L of iso-α-acid substrate (Isolone® Isomerized Hop Extract Solution, Kalsec), and 0.02 g/L NADP in 40 vol% isopropanol (IPA) in 100 mM pH 8 potassium phosphate with 2 mM MgSO₄. The plates were sealed and incubated at 45° C. with shaking at 600 rpm for 20-24 hours.

After 20-24 hours, 1000 µL of acetonitrile with 0.1% acetic acid was added. The plates were sealed and centrifuged at 4000 rpm at 4° C. for 10 min. The quenched sample was further diluted 20-40x in 50:50 acetonitrile:water mixture prior to HPLC analysis. The HPLC run parameters are described in Table 5-2.

TABLE 7-1

KRED Variant Activity Relative to SEQ ID NO: 80

| SEQ ID NO: (nt/aa) | Amino Acid Differences (Relative to SEQ ID NO: 80) | Percent Conversion Fold Improvement (Relative to SEQ ID NO: 80)¹ |
|---|---|---|
| 99/100 | L211R | ++++ |
| 101/102 | L17S | ++++ |
| 103/104 | L17Q | +++ |
| 105/106 | D198A | +++ |
| 107/108 | T152K | +++ |
| 109/110 | D101T | +++ |
| 111/112 | I110V | +++ |
| 113/114 | D101C | ++ |
| 115/116 | P190A | ++ |
| 117/118 | V56C | ++ |
| 119/120 | A162G | ++ |
| 121/122 | V95I | ++ |
| 123/124 | T210W | ++ |
| 125/126 | T210F | ++ |
| 127/128 | L21A | ++ |
| 129/130 | C227V | + |
| 131/132 | K46V | + |
| 133/134 | D101L | + |
| 135/136 | D198Q | + |
| 137/138 | T152L | + |
| 139/140 | E79L | + |
| 141/142 | K72A | + |

¹Levels of increased activity were determined relative to the reference polypeptide of SEQ ID NO: 80 and defined as follows: “+” >1.0 but < 2.0, “++” ≥2 but ≤ 4, “+++” ≥ 4 but ≤ 8, “++++” ≥ 8

### Example 8

### Evolution and Screening of Engineered Polypeptides Derived From SEQ ID NO: 80 for Improved KRED Activity

Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 80 (i.e., SEQ ID NO: 79), was used to generate the further engineered polypeptides of Table 8-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 80 using directed evolution methods as described above together with the HTP assay and analytical methods described below in Table 5-2.

Directed evolution began with the polynucleotide set forth in SEQ ID NO: 79. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acids products.

The enzyme assay was carried out in a 96-well format, in 200 µL total volume/well, which included 10-20% v/v HTP enzyme lysate, 80 or 160 g/L of iso-α-acid substrate (Isolone® Isomerized Hop Extract Solution, Kalsec), and 0.02 g/L NADP in 40 vol% isopropanol (IPA) in 100 mM pH 8 potassium phosphate with 2 mM MgSO₄. The plates were sealed and incubated at 45° C. with shaking at 600 rpm for 20-24 hours.

After 20-24 hours, 1000 µL of acetonitrile with 0.1% acetic acid was added. The plates were sealed and centrifuged at 4000 rpm at 4° C. for 10 min. The quenched sample was further diluted 20-40x in 50:50 acetonitrile:water mixture prior to HPLC analysis. The HPLC run parameters are described in Table 5-2.

TABLE 8-1

KRED Variant Activity Relative to SEQ ID NO: 80

| SEQ ID NO: (nt/aa) | Amino Acid Differences (Relative to SEQ ID NO: 80) | Percent Conversion Fold Improvement (Relative to SEQ ID NO: 80)¹ |
|---|---|---|
| 143/144 | P190A;P194E | ++++ |
| 145/146 | P194E | ++++ |
| 147/148 | N157C | ++++ |
| 149/150 | L17M | ++++ |
| 99/100 | L211R | ++++ |
| 151/152 | L17S | +++ |
| 153/154 | I191T;P194E | +++ |
| 155/156 | P190A;I191T;P194E | +++ |
| 103/104 | L17Q | ++ |
| 157/158 | S159T | ++ |
| 159/160 | D198Q | ++ |
| 139/140 | E79L | + |
| 161/162 | D198A | + |

¹Levels of increased activity were determined relative to the reference polypeptide of SEQ ID NO: 80 and defined as follows: “+” >1.0 but < 2.0, “++” ≥2 but ≤ 4, “+++” ≥ 4 but ≤ 8, “++++” ≥ 8

### Example 9

### Evolution and Screening of Engineered Polypeptides Derived From SEQ ID NO: 6 and SEQ ID NO: 80 for Improved KRED Activity at High Substrate Concentration

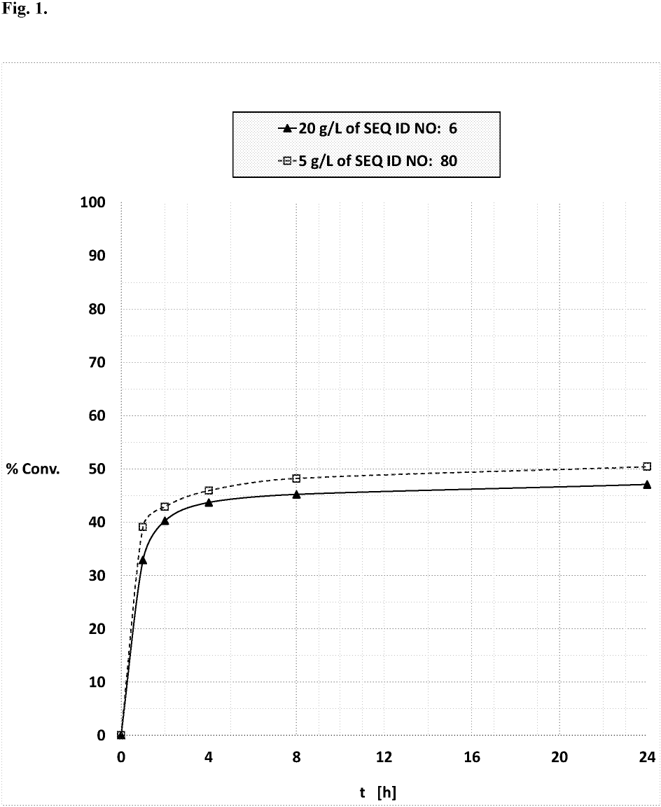

A 40 g/L enzyme stock solution was prepared by dissolving 200 mg of enzyme powder in 5 mL of 100 mM pH 8 triethanolamine∗H₂SO₄ with 2 mM MgSO₄. A 2 mL aliquot was taken and subjected to two successive 1:1 v/v dilution to each 20 and 10 g/L. 500 µL of enzyme stock solution (10, 20 or 40 g/L) were added to a vial under air with stir bar. To the stirred enzyme stock solution was added a 100 µL aliquot of 1 g/L NADP in buffer and 400 µL of 25 g/L iso-α-acids in isopropanol (IPA). The final reaction composition was 5, 10, or 20 g/L enzyme, 10 g/L iso-α-acids, and 0.1 g/L NADP in 40% IPA. The vial was placed in a heating block at 25° C. or 40° C. and sampled after 1, 2, 4, 8, and 24 h and analyzed by HPLC-UV. A typical reaction profile comparing SEQ ID NO: 6 and SEQ ID NO: 80 is shown in FIG. 1.

### Example 10

### Evolution and Screening of Engineered Polypeptides Derived From SEQ ID NO: 80, 104, 100, 136,116,132,162,150,152,144 and 146 for Improved KRED Activity at High Substrate and Low NADP Concentration

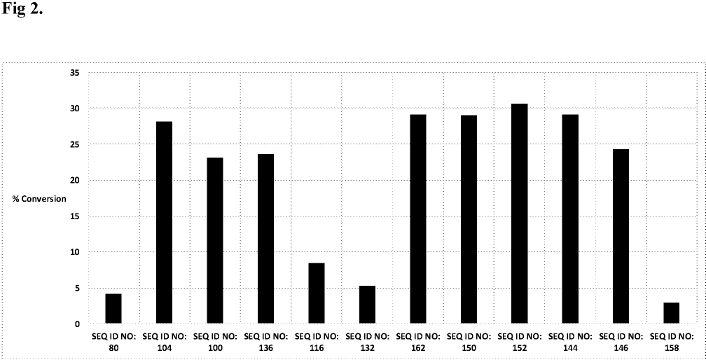

A 200 g/L enzyme stock solution was prepared by dissolving 100 mg of enzyme powder in 500 µL of 100 mM pH 8 potassium phosphate buffer with 2 mM MgSO₄ and 0.1 g/L of NADP. To a well in a 96 deep-well plate were added 40 µL of the enzyme/NADP stock solution, 80 µL of isopropanol, and 80 µL of 40 wt% aqueous solution of iso-α-acids. The final reaction composition was 40 g/L of enzyme, 160 g/L iso-α-acids, and 0.02 g/L NADP in 40% IPA. The plate was sealed and incubated at 40° C. for 24 h and then quenched and analyzed by HPLC-UV. The data are shown in Table 11-1 and depicted in FIG. 2.

TABLE 10-1

KRED Activity at High Substrate and Low NADPH Concentration

| SEQ ID NO: (nt/aa) | % Conversion | | | | | |
|---|---|---|---|---|---|---|
| | 40 g/L | 20 g/L | 10 g/L | 5 g/L | 2.5 g/L | 1.25 g/L |
| 79/80 | 4.2 | 1.9 | 0.9 | 0.5 | 0.1 | 0.0 |
| 103/104 | 28.2 | 16.5 | 8.7 | 5.2 | 2.2 | 1.2 |
| 99/100 | 23.1 | 11.2 | 6.1 | 3.3 | 1.3 | 0.6 |
| 135/136 | 23.6 | 7.5 | 2.4 | 1.2 | 0.6 | 0.0 |
| 115/116 | 8.5 | 3.2 | 1.2 | 0.7 | 0.2 | 0.0 |
| 131/132 | 5.3 | 2.2 | 0.8 | 0.4 | 0.1 | 0.0 |
| 161/162 | 29.1 | 14.4 | 5.6 | 2.1 | 0.7 | 0.3 |
| 149/150 | 29.0 | 14.9 | 6.0 | 2.4 | 1.0 | 0.2 |
| 151/152 | 30.6 | 17.9 | 7.4 | 3.6 | 2.0 | 1.2 |
| 143/144 | 29.1 | 14.4 | 5.8 | 2.4 | 1.2 | 0.4 |
| 145/146 | 24.3 | 12.3 | 4.7 | 1.9 | 0.8 | 0.1 |
| 157/158 | 3.0 | 1.1 | 0.4 | 0.0 | 0.0 | 0.0 |

### Example 11

### Evolution and Screening of Engineered Polypeptides Derived From SEQ ID NO: 104 for Improved KRED Activity

As described in Example 8, libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 104 (i.e., SEQ ID NO: 103), was used to generate the further improved, engineered polypeptides of Table 11-1.

TABLE 11-1

KRED Variant Product Conversion Relative to SEQ ID NO: 104

| SEQ ID NO: (nt/aa) | Amino Acid Differences (Relative to SEQ ID NO: 104) | Percent Conversion Fold Improvement (Relative to SEQ ID NO: 104)¹ |
|---|---|---|
| 163/164 | Q17M/P190A/D198A | + |
| 165/166 | Q17M/K46V/D 198A/L211R | + |
| 167/168 | K46V/P194E/D198Q | + |
| 169/170 | Q17M/K46V/P190A | + |
| 171/172 | Q17M/P190A/D198Q | ++ |
| 173/174 | K46V/P190A/P194E/D 198Q | + |
| 175/176 | Q17M/I96V/P194E/D198Q | + |

¹levels of increased conversion were determined relative to the reference polypeptide of SEQ ID NO: 104 and defined as follows: “+” ≥ 4.0, “++” ≥8.0

### Example 12

### E. Coli Expression Hosts Containing Recombinant KRED Genes

The initial KRED enzymes used to produce the variants of the present invention were obtained from Codexis’s collection of commercially available KRED enzyme panels. During the initial screen, the polypeptide of SEQ ID NO: 172 or polypeptide of SEQ ID NO: 270 produced the most product as determined by LC/MS. The KRED-encoding genes were cloned into the expression vector of SEQ ID NO: 413 or SEQ ID NO: 414, operatively linked to the lac promoter under control of the lacl repressor. The expression vector also contains the P15a origin of replication and a chloramphenicol resistance gene. The resulting plasmids were transformed into E. coli W3110, using standard methods known in the art. The transformants were isolated by subjecting the cells to triclosan selection, as known in the art (See e.g., U.S. Pat. No. 8,383,346 and WO2010/144103).

### Example 13

### Preparation of HTP KRED-Containing Wet Cell Pellets

E. coli cells containing recombinant KRED-encoding genes from monoclonal colonies were inoculated into 190 µl LB containing 1% glucose and 0.12 µg/mL of triclosan in the wells of 96-well shallow-well microtiter plates. The plates were sealed with O₂-permeable seals, and cultures were grown overnight at 20° C., 200 rpm, and 85% humidity. Then, 20 µl of each of the cell cultures were transferred into the wells of 96-well deep-well plates containing 380 µL TB and 30 µg/mL CAM. The deep-well plates were sealed with O₂-permeable seals and incubated at 30° C., 250 rpm, and 85% humidity until an OD₆₀₀ of 0.6-0.8 was reached. The cell cultures were then induced by IPTG to a final concentration of 1 mM and incubated overnight under the same conditions as originally used. The cells were then pelleted using centrifugation at 4° C., 4000 rpm for 10 min. The supernatants were discarded, and the pellets were frozen at -80° C. prior to lysis.

### Example 14

### Preparation of HTP KRED-Containing Cell Lysates

First, the cell pellets that were produced as described in Example 2 were lysed by adding 150 µL lysis buffer containing 100 mM, pH 8 potassium phosphate with 2 mM MgSO₄ or 100 mM, pH 8 potassium phosphate with 2 mM MgSO₄, 1 g/L lysozyme, and 0.5 g/L PMBS. Then, the cell pellets were shaken at room temperature for 2 hours on a bench top shaker. The plates were centrifuged at 4,000 rpm, for 15 minutes at 4° C. to remove cell debris. The supernatants were then used in biocatalytic reactions to determine their activity levels.

### Example 15

### Preparation of Lyophilized Lysates From Shake Flask (SF) Cultures

Shake-flask procedures can be used to generate engineered KRED polypeptide shake-flask powders (SFP), which are useful for secondary screening assays and/or use in the biocatalytic processes described herein. Shake flask powder (SFP) preparation of enzymes provides a more purified preparation (e.g., up to 30% of total protein) of the engineered enzyme, as compared to the cell lysate used in HTP assays and also allows for the use of more concentrated enzyme solutions. To start this, selected HTP cultures grown as described above were plated onto LB agar plates with 1% glucose and 0.12 µg/mL of triclosan, and grown overnight at 37° C. A single colony from each culture was transferred to 6 ml of LB with 1% glucose and 30 µg/ml CAM. The cultures were grown for 18 h at 30° C. at 250 rpm and subcultured approximately 1:50 into 250 ml of TB containing 0.12 µg/mL of triclosan, to a final OD₆₀₀ of 0.05. The cultures were grown for approximately 3 hours at 30° C. at 250 rpm to an OD₆₀₀ between 0.8-1.0 and induced with 1 mM IPTG. The cultures were then grown for 20 h at 30° C. at 250 rpm. The cultures were centrifuged (4,000 rpm for 20 min at 4° C.). The supernatant was discarded, and the pellets were re-suspended in 35 ml of 50 mM, pH 8 potassium phosphate with 2 mM MgSO₄. The re-suspended cells were centrifuged (4,000 rpm for 20 min at 4° C.). The supernatant was discarded, and the pellets were re-suspended in 6 ml of 50 mM, pH 8 potassium phosphate with 2 mM MgSO₄, and the cells were lysed using a cell disruptor from Constant Systems (One Shot). The lysates were pelleted (10,000 rpm for 60 min at 4° C.), and the supernatants were frozen and lyophilized to generate shake flake (SF) enzymes.

### Example 16

### Evolution and Screening of Engineered Polypeptides Derived From SEQ ID NO: 172 for Improved KRED Activity

The polypeptide of SEQ ID NO: 172 was selected as the parent enzyme based on the results of screening variants for the reduction of the iso-α-acid substrate. Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 172 was used to generate the further engineered polypeptides of Table 16-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 172 using directed evolution methods as described above together with the HTP assay and analytical methods described below in Table 16-1.

Directed evolution began with the polynucleotide set forth in SEQ ID NO: 171. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-deep well plate format, in 100 µL total volume/well, which included 20% v/v HTP enzyme lysate, 40% v/v of 40 wt% aqueous solution of iso-α-acid substrate (Isolone® Isomerized Hop Extract Solution, Kalsec), and 0.02 g/L NADP in 40 vol% isopropanol (IPA) in 100 mM, pH 8 potassium phosphate with 2 mM MgSO_{4.} The plates were sealed and incubated at 45° C. with shaking at 600 rpm for 20-24 hours.

After 20-24 hours, 1000 µL of acetonitrile with 0.1% acetic acid were added. The plates were sealed and centrifuged at 4,000 rpm at 4° C. for 10 min. The quenched sample was further diluted 4-5x in 50:50 acetonitrile:water mixture prior to HPLC analysis. The HPLC run parameters are below in Table 5-2.

TABLE 16-1

KRED Variant Activity Relative to SEQ ID NO:172

| SEQ ID NO: (nt/aa) | Amino Acid Differences (Relative to SEQ ID NO:172) | Percent Conversion Fold Improvement (Relative to SEQ ID NO:172)¹ |
|---|---|---|
| 177/178 | E204Q | + |
| 179/180 | I226V | + |
| 181/182 | D101Y | + |
| 183/184 | Y179M | + |
| 185/186 | D101R | ++ |
| 187/188 | A231G | + |
| 189/190 | P194E | + |
| 191/192 | E45L | + |

¹levels of increased activity were determined relative to the reference polypeptide of SEQ ID NO: 172 and defined as follows: “+” >1.0 but < 1.5, “++” ≥1.5

### Example 17

### Evolution and Screening of Engineered Polypeptides Derived From SEQ ID NO: 186 for Improved KRED Activity on Trans-ISO

SEQ ID NO: 186 was selected as the parent enzyme based on the results of screening variants for the reduction of the iso-α-acid substrate. Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 185 was used to generate the further engineered polypeptides of Table 17-2. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 186 using directed evolution methods as described above together with the HTP assay and analytical methods described in Table 5-2.

Directed evolution began with the polynucleotide set forth in SEQ ID NO: 185. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-deep well plate format, in 100 µL total volume/well, which included 50% v/v HTP enzyme lysate, 10% v/v of 10 g/L of SEQ ID NO: 186, 1 g/L iso-α-acid substrate (Isolone® Isomerized Hop Extract Solution, Kalsec), and 0.1 g/L NADP in 40 vol% isopropanol (IPA) in 100 mM, pH 8 potassium phosphate with 2 mM MgSO₄. The plates were sealed and incubated at 30° C. with shaking at 600 rpm for 44-48 hours.

After 20-24 hours, 1000 µL of acetonitrile with 0.1% acetic acid were added. The plates were sealed and centrifuged at 4,000 rpm at 4° C. for 10 min. The quenched sample was further diluted 4-5x in 50:50 acetonitrile:water mixture prior to HPLC analysis. The HPLC run parameters are described below in Table 17-1. See FIG. 3. Variants that showed improved activity towards the Trans-ISOs are shown in Table 17-2.

TABLE 17-1

HPLC Parameters

| Instrument | Agilent 1100 HPLC | | |
|---|---|---|---|
| Column | 3 × 150 mm 2.1 µm Waters Atlantis T3 column | | |
| Mobile Phase | 30% acetonitrile in 50 mM pH 8 potassium phosphate | | |
| Flow Rate | 0.8 mL/min | | |
| Run time | 20 min | | |
| | Compound | retention time [min] | note |
| Peak Retention Times | Rho-1 | 3.4 | trans-co-Rho |
| | Rho-2 | 4.8 | cis-co-Rho |
| | Rho-3 | 5.4 | trans-n/ad-Rho |
| | Rho-4 | 7.0 | mixture of Rho isomers |
| | Rho-5 | 7.3 | mixture of Rho isomers |
| | Rho-6 | 7.7 | cis-n/ad-Rho |
| | ISO-1 | 9.0 | cis-co-ISO |
| | ISO-2 | 10.2 | trans-co-ISO |
| | Rho-7 | 12.5 | trans-n/ad-Rho |
| | ISO-3 | 15.1 | cis-n/ad-ISO |
| | ISO-4 | 17.4 | trans-n/ad-ISO |
| Column Temperature | 40° C. | | |
| Injection Volume | 10 µL | | |
| Detection | 270 nm | | |

TABLE 17-2

KRED Variant Activity Relative with improved trans-ISO activity

| SEQ ID NO: (nt/aa) | Amino Acid Differences (Relative to SEQ ID NO:186) | Activity on Trans-ISO¹ |
|---|---|---|
| 193/194 | D150A/L153A/M205A/L211A | + |
| 195/196 | D150A/L153A/L211A | + |
| 197/198 | V95A/K97A/D150A/L153A | + |
| 199/200 | V95A/D150A/L153A/M205A/L211A | + |
| 201/202 | K97A/D150A/L153A | + |
| 203/204 | M145A/L153A/L211A | + |
| 205/206 | I96A/D150A/L153A | + |
| 207/208 | K97A/D150A/L153A/M205A/L211A | + |
| 209/210 | V95A/D15OA/L153A/M205A/M206A/L211A | + |
| 211/212 | K97A/D150A/L153A/M205A | + |
| 213/214 | D150A/L153A/M206A/L211A | + |
| 215/216 | D150A/L153A/M206A/L211A | + |
| 217/218 | V95A/I96A/K97A/D150A/L153A/M205A | + |
| 219/220 | V95A/K97A/S143A/M145A/D150A/L153A/W202A/M205A | + |
| 221/222 | K97A/D150A/L153A/M206A | + |
| 223/224 | V95A/K97A/D150A/L153A/W202A/M205A/M206A | + |
| 225/226 | S143A/I144A/M145A/D150A/L153A/W202A/M205A/W249A | + |
| 227/228 | S143A/M145A/D150A/L153A | + |
| 229/230 | V95A/K97A/S143A/M145A/D150A/L153A/W249A | + |
| 231/232 | I96A/D150A/L153A/M206A | + |
| 233/234 | I144A/D150A/L153A/W202A/M205A/M206A | + |
| 235/236 | V95A/I96A/D150A/L153A/M205A/M206A/L211A/W249A | + |
| 237/238 | V95A/D150A/L153A/M206A/W249A | + |
| 239/240 | I144A/M145A/D150A/L153A/M205A/M206A | + |
| 241/242 | M145A/D150A/L153A/M206A/W249A | + |
| 243/244 | D150A/L153A/W249A | + |
| 245/246 | I144A/D150A/L153A | + |
| 247/248 | D150A/L153A/W202A/M206A/W249A | + |
| 249/250 | V95A/D150A/L153A/M206A/W249A | + |

¹ SEQ ID NO: 186 showed no detectable activity on trans-ISO

### Example 18

### Evolution and Screening of Engineered Polypeptides Derived From SEQ ID NO:186 for Improved KRED Activity

As described in Example 16, libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 186 (i.e., SEQ ID NO: 185), was used to generate the further improved, engineered polypeptides of Table 18-1.

TABLE 18-1

KRED Variant Activity Relative to SEQ ID NO:186

| SEQ ID NO: (nt/aa) | Amino Acid Differences (Relative to SEQ ID NO: SEQ ID NO:186) | Percent Conversion Fold Improvement (Relative SEQ ID NO:186)¹ |
|---|---|---|
| 251/252 | I110V/Y179M/P194E | ++ |
| 253/254 | I110V | ++ |
| 255/256 | T103R/L147I | ++ |
| 257/258 | I110V | ++ |
| 259/260 | H7Q/L147I | + |
| 261/262 | W249Y | + |
| 263/264 | L147I | + |

¹Levels of increased activity were determined relative to the reference polypeptide of SEQ ID NO:186 and defined as follows: “+” >1.2 but < 1.5, “++” ≥1.5

### Example 19

### Evolution and Screening of Engineered Polypeptides Derived From SEQ ID NO: 194, SEQ ID NO: 252 for Improved KRED Activity at High Substrate and Low NADP Concentration

A 200 g/L enzyme stock solution was prepared by dissolving 100 mg of enzyme powder in 500 µL of 100 mM, pH 8 potassium phosphate buffer with 2 mM MgSO₄ and 0.1 g/L of NADP. To a well in a 96 deep-well plate was added a 40 µL aliquot of the enzyme/NADP stock solution, 80 µL of isopropanol, and 80 µL of 40 wt% aqueous solution of iso-α-acids. The final reaction composition was 40 g/L of enzyme, 160 g/L iso-α-acids, and 0.02 g/L NADP in 40% IPA. The plate was sealed and incubated at 40° C. for 24 h and then quenched and analyzed by HPLC-UV. The data are shown in Tables 19-1, 19-2 and 19-3.

TABLE 19-1

KRED Variant Activity Relative to SEQ ID NO:194

| SEQ ID NO: (nt/aa) | Amino Acid Differences (Relative to SEQ ID NO:194) | Percent Conversion Fold Improvement (SEQ ID NO:194) ¹ |
|---|---|---|
| 265/266 | V12I/K72S/I110V/L147I/T152M/E204Q | ++ |
| 267/268 | V12I/K72S/R101Y/T103Q/I110V/T152M/W249Y | ++ |
| 269/270 | E45L/T54S/K72S/I110V/T152M/P194E/E204Q | ++ |
| 271/272 | V12I/T54S/K72T/I110V/A150D/T152M/A153L/P194E/A205M/A211L/W249Y | ++ |
| 273/274 | I110V/A150D/A153L/Y179M/P194E/A205M/A211L/W249Y | + |
| 275/276 | H7Q/V12I/T54S/I110V/A150D/A153L/P194E/A205M/A211L/W249Y | + |
| 277/278 | K72S/I110V/L147M/A150D/T152M/A153L/PI194E/A205M/A211L/W249Y | + |

¹levels of increased activity were determined relative to the reference polypeptide of SEQ ID NO:194 and defined as follows: “+” >1.2 but < 1.5, “++” ≥1.5

TABLE 19-2

KRED Variant Activity Relative to SEQ ID NO: 252

| SEQ ID NO: (nt/aa) | Amino Acid Differences (Relative to SEQ ID NO: 252) | Percent Conversion Fold Improvement (Relative to SEQ ID NO: 252)¹ |
|---|---|---|
| 277/278 | K72S/L147M/T152M/M179Y/W249Y | +++ |
| 275/276 | H7Q/V12I/T54S/M 179Y/W249Y | +++ |
| 271/272 | V12I/T54S/K72T/T152M/M179Y/W249Y | ++++ |
| 273/274 | W249Y | ++ |
| 279/280 | H40E | + |
| 281/282 | T54S/K72S | + |
| 283/284 | H7Q/T152M | ++ |

¹Levels of increased activity were determined relative to the reference polypeptide of SEQ ID NO: 252 and defined as follows: “+” >1.2 but < 1.5, “++” >1.5, “+++” >1.5 but < 2.0; “++++” > 2.0

TABLE 19-3

KRED Activity at High Substrate and Low NADPH Concentration

| SEQ ID NO: (nt/aa) | % Conversion | | | | | |
|---|---|---|---|---|---|---|
| | 40 g/L | 20 g/L | 10 g/L | 5 g/L | 2.5 g/L | 1.25 g/L |
| 193/194 | 43 | 26 | 17 | 7 | 2 | 1 |
| 251/252 | 42 | 30 | 23 | 12 | 5 | 1 |
| 269/270 | 47 | 30 | 22 | 11 | 5 | 2 |
| 271/272 | 48 | 33 | 31 | 25 | 14 | 7 |

### Example 20

### Evolution and Screening of Engineered Polypeptides Derived From SEQ ID NO:270 and SEQ ID NO: 272 for Improved KRED Activity at High Substrate and Low NADP Concentration

As described in Example 18, libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotides encoding the polypeptides having KRED activity of SEQ ID NO: 270 (i.e., SEQ ID NO: 269) and NO: 272 (i.e., SEQ ID NO: 271), were used to generate the further improved, engineered polypeptides of Tables 20-1 and 20-2.

TABLE 20-1

KRED Variant Activity Relative to SEQ ID NO:270

| SEQ ID NO: (nt/aa) | Amino Acid Differences (Relative to SEQ ID NO:270) | Percent Conversion Fold Improvement (Relative to SEQ ID NO:270)¹ |
|---|---|---|
| 285/286 | A150Y/M152A | ++ |
| 287/288 | A150Y/M152S | ++ |
| 289/290 | E194S/R195A | + |
| 291/292 | G92A/I93E | + |
| 293/294 | A150D/M152A/A153L | + |

¹Levels of increased activity were determined relative to the reference polypeptide of SEQ ID NO:270 and defined as follows: “+” >1.2 but < 1.5, “++” ≥1.5

TABLE 20-2

KRED Variant Activity Relative to SEQ ID NO: 272

| SEQ ID NO: (nt/aa) | Amino Acid Differences (Relative to SEQ ID NO: 272) | Percent Conversion Fold Improvement (Relative to SEQ ID NO: 272)¹ |
|---|---|---|
| 295/296 | I93D/V95R | +++ |
| 297/298 | I93A/V95K/K109R | ++++ |
| 299/300 | I93A/V95R/K109D/N114T | ++++ |
| 301/302 | I93R/V95A/N114T | +++ |
| 303/304 | I93M | ++ |
| 305/306 | I93E/K109R/N114A | ++ |
| 307/308 | N114A | + |
| 309/310 | G92A/I93D/V95R | ++ |

¹Levels of increased activity were determined relative to the reference polypeptide of SEQ ID NO: 272 and defined as follows: “+” >1.2 but < 1.5, “++” >1.5, “+++” >1.5 but < 2.0, “++++” >2.0

### Example 21

### Evolution and Screening of Engineered Polypeptides Derived From SEQ ID NO: 286 for Improved KRED Activity at High Substrate and Low NADP Concentration

As described in Example 18, libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 286 (i.e., SEQ ID NO: 285), were used to generate the further improved, engineered polypeptides of Table 21-1.

TABLE 21-1

KRED Variant Activity Relative to SEQ ID NO: 286

| SEQ ID NO: (nt/aa) | Amino Acid Differences (Relative to SEQ ID NO: 286) | Percent Conversion Fold Improvement (Relative to SEQ ID NO: 286)¹ |
|---|---|---|
| 311/312 | I96A | + |
| 313/314 | M145A/Y150A | + |
| 315/316 | V12I/L45E/W249Y | ++ |
| 317/318 | L45E/W249Y | ++ |
| 319/320 | L45E/K109D/W249Y | +++ |
| 321/322 | L45E/S72T/W249Y | ++ |
| 323/324 | V12I/L45E/I93A/W249Y | ++ |
| 325/326 | V12I/K109D/W249Y | ++ |
| 327/328 | V12I/L45E/S72T/K109D/W249Y | +++ |

¹Levels of increased activity were determined relative to the reference polypeptide of SEQ ID NO: 286 and defined as follows: “+” >1.2 but < 1.5, “++” >1.5, “+++” >1.5 but < 2.0, “++++” >2.0

### Example 22

### Evolution and Screening of Engineered Polypeptides Derived From SEQ ID NO: 328 for Improved KRED Activity

SEQ ID NO: 328 was selected as the parent enzyme based on the results of screening variants for the reduction of the iso-α-acid substrate. Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 327 was used to generate the further engineered polypeptides of Table 22-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 328 using directed evolution methods as described above together with the HTP assay and analytical methods described in Table 5-2.

Directed evolution began with the polynucleotide set forth in SEQ ID NO: 327. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-well round bottom plate, in 200 µL total volume/well, which included 20% v/v HTP enzyme lysate, 40% v/v of 40 wt% aqueous solution of iso-α-acid substrate (Isolone® Isomerized Hop Extract Solution, Kalsec), and 0.02 g/L NADP in 40 vol% isopropanol (IPA) in 100 mM, pH 8 potassium phosphate with 2 mM MgSO_{4.} The plates were sealed and incubated at 45° C. with shaking at 600 rpm for 20-24 hours.

After 20-24 hours, 1000 µL of acetonitrile with 0.1% acetic acid were added. The plates were sealed and centrifuged at 4,000 rpm at 4° C. for 10 min. The quenched sample was further diluted 4-5x in 50:50 acetonitrile:water mixture prior to HPLC analysis. The HPLC run parameters are described in Table 5-2 and Table 17-1. The improved variants are shown in Table 22-1.

TABLE 22-1

KRED Variant Activity Relative to SEQ ID NO: 328

| SEQ ID NO: (nt/aa) | Amino Acid Differences (Relative to SEQ ID NO: 328) | Percent Conversion Fold Improvement (Relative to SEQ ID NO: 328)¹ |
|---|---|---|
| 329/330 | Y150A/R195S | +++ |
| 331/332 | Y150A | +++ |
| 333/334 | R195S | ++ |
| 335/336 | R195A | + |
| 337/338 | R195S | + |
| 339/340 | Y150A/P151A | + |
| 341/342 | Y150A/R195S | + |

¹Levels of increased activity were determined relative to the reference polypeptide of SEQ ID NO: 328 and defined as follows: “+” >1.2 but < 1.5, “++” >1.5, “+++” >1.5 but < 2.0, “++++” >2.0

### Example 23

### Performance of SEQ ID NO: 328 and SEQ ID NO: 330 at High Substrate and Low NADP Loadings

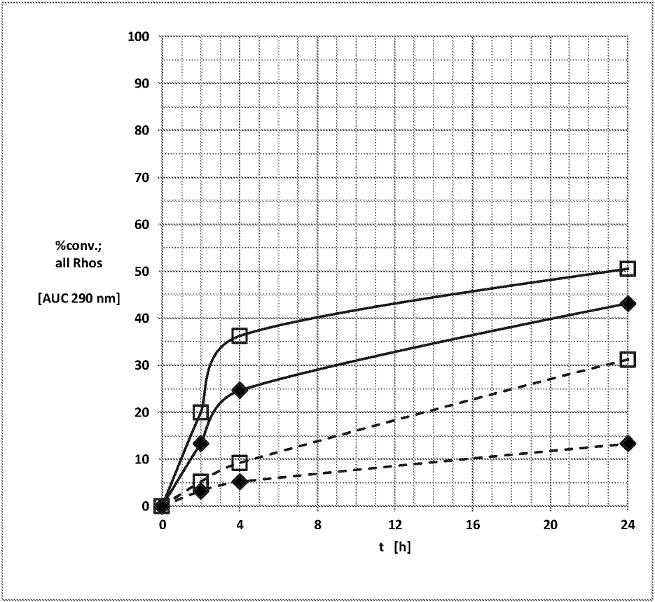

A sample of 8 g of 40 wt% aqueous ISO solution was dissolved in 8 mL of isopropanol (IPA) and 2 mL of 100 mM, pH 8 potassium phosphate buffer. The pH was adjusted to 8 with 4 N NaOH and/or 10% H₃PO₄. 4.5 mL of the pH-adjusted ISO/IPA/buffer solution were added to septum-capped vials with a stir bar and under a nitrogen blanked in a heating block at 40° C. 0.5 mL of 10 or 40 g/L of KRED in 100 mM, pH 8 potassium phosphate buffer with 10 mM of MgSO₄ and 0.2 g/L of NADP were added to the solutions (final KRED and NADP loadings are 1 or 4 g/L KRED and 0.02 g/L NADP respectively). At various time intervals, 20 µL aliquots were withdrawn from the reaction via a syringe, quenched with 1000 µL of 1:1 acetonitrile/water with 0.1% acetic acid. After centrifugation at 4,000 rpm at 20° C. for 5 min, 20 µL of the supernatants were diluted with 180 µL of 1:1 acetonitrile/water with 0.1% acetic acid for HPLC analysis (Table 5-2 and Table 17-1). See FIG. 4.

### Example 24

### Evolution and Screening of Engineered Polypeptides Derived From SEQ ID NO: 330 for Improved KRED Activity

SEQ ID NO: 330 was selected as the parent enzyme based on the results of screening variants for the reduction of the iso-α-acid substrate. Libraries of engineered genes were produced using well-established techniques (e.g., saturation mutagenesis, and recombination of previously identified beneficial mutations). The polypeptides encoded by each gene were produced in HTP as described in Example 2, and the soluble lysate was generated as described in Example 3.

The engineered polynucleotide encoding the polypeptide having KRED activity of SEQ ID NO: 329 was used to generate the further engineered polypeptides of Table 24-1. These polypeptides displayed improved formation of dihydro-(rho)-iso-α-acids from iso-α-acids as compared to the starting polypeptide. The engineered polypeptides were generated from the “backbone” amino acid sequence of SEQ ID NO: 330 using directed evolution methods as described above together with the HTP assay and analytical methods described below in Table 24-1.

Directed evolution began with the polynucleotide set forth in SEQ ID NO: 329. Engineered polypeptides were then selected as starting “backbone” gene sequences. Libraries of engineered polypeptides were generated using various well-known techniques (e.g., saturation mutagenesis, recombination of previously identified beneficial amino acid differences) and screened using HTP assay and analysis methods that measured the polypeptides’ ability to convert the iso-α-acid substrates to the desired dihydro-(rho)-iso-α-acid product.

The enzyme assay was carried out in a 96-round bottom plate format, in 200 µL total volume/well, which included 50% v/v HTP enzyme lysate, 10% v/v of 40 wt% aqueous solution of iso-α-acid substrate (Isolone® Isomerized Hop Extract Solution, Kalsec), and 0.02 g/L NADP in 40 vol% isopropanol (IPA) in 100 mM, pH 8 potassium phosphate with 2 mM MgSO₄. The plates were sealed and incubated at 45° C. with shaking at 600 rpm for 20-24 hours.

TABLE 24-1

KRED Variant Activity Relative to SEQ ID NO: 330

| SEQ ID NO: (nt/aa) | Amino Acid Differences (Relative to SEQ ID NO: 330) | Percent Conversion Fold Improvement (Relative to SEQ ID NO: 330)¹ |
|---|---|---|
| 343/344 | V95R/S195R | ++ |
| 345/346 | I93A/S 195R | ++ |
| 347/348 | I93A/V95R/M145A/S195R | + |
| 349/350 | I93A/D109K/N114T/M145A/S195R | ++ |
| 351/352 | M145A/S195A | + |
| 353/354 | S195R | + |
| 355/356 | T72K/A152M/S195R | ++ |
| 357/358 | I12V/T72S/D109K/S195R | ++ |
| 359/360 | T72S/D109K/A152M/S195R | ++ |
| 361/362 | T72S/D109K/S195R | ++ |
| 363/364 | T72K/S195R | + |
| 365/366 | I93A | + |
| 367/368 | E194N | + |
| 369/370 | E194N/E200P | + |
| 371/372 | E200P | + |
| 373/374 | E79A | + |
| 375/376 | I96P/E194N/E200P | + |
| 377/378 | I96P/R108S/L147I/E200P | + |
| 379/380 | K192R | + |
| 381/382 | L147I | + |
| 383/384 | L147I/E200P | + |
| 385/386 | L73V | + |
| 387/388 | L73V/L147I | + |
| 389/390 | M17A/L115Q | + |
| 391/392 | M17A/L73V/E200P | + |
| 393/394 | Q198G | + |
| 395/396 | Q198R | + |
| 397/398 | A68R/T72D/R101Q/A152Q/A205L | + |
| 399/400 | A68E/T72D/R101K/A152Q/A205L | + |
| 401/402 | R101M/A205L | + |
| 403/404 | A68R/T72R/L124E | + |
| 405/406 | A68R/T72R/L124E/A152Q | + |
| 407/408 | T72D/A152Q | + |
| 409/410 | A68R/L124E/A205L | + |
| 411/412 | A68R/R101Q/L124E/A152Q/A205L | + |

¹Levels of increased activity were determined relative to the reference polypeptide of SEQ ID NO: 330 and defined as follows: “+” >1.2 but < 1.5, “++” >1.5, “+++” >1.5 but < 2.0, “++++” >2.0

### Example 25

### Performance of SEQ ID NO:270, SEQ ID NO: 328, SEQ ID NO: 330, SEQ ID NO: 348, SEQ ID NO: 346 and SEQ ID NO: 356 at High Substrate and Low NADP Loadings

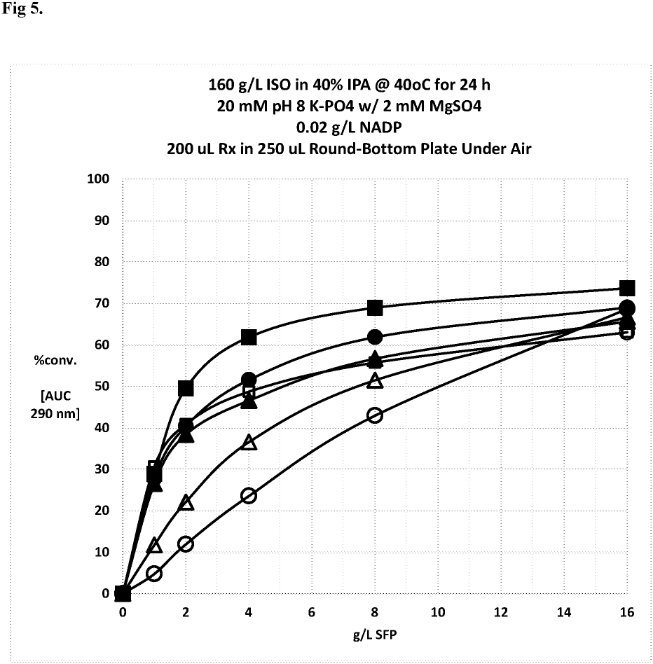

A sample of 80 mg of each variant was dissolved in 1 mL of NADP stock solution made up with 10 mg of NADP in 100 mL of 100 mM, pH 9 potassium phosphate and 10 mM of MgSO₄. The stock solution was subjected to 5 successive 1:1 dilutions to give stock solution of 80, 40, 20, 10 and 5 g/L enzyme in NADP stock solution. A 20 uL aliquot of the stock solution was added to 40 uL of 40 wt% ISO solution and 40 uL of IPA in a round-bottom plate to give a final composition of 160 g/L of ISO in 40 vol% of IPA in 20 mM, pH 8 potassium phosphate and 2 mM MgSO₄ with 0.02 g/L of NADP. The final enzyme concentrations were 16, 8, 4, 2 and 1 g/L respectively. The plates were sealed and placed in a shaker at 40° and 600 rpm for 24 hours. After 24 hours, the plates were centrifuged at 4,000 rpm at 20° C. for 10 minutes, and 100 ul of the supernatant were transferred to 1 mL of: 1 acetonitrile/water with 0.1% acetic acid in a deep-well plate. The deep-well plates with the quenched reaction mixture were centrifuged at 4,000 rpm at 20° C. for 10 minutes, and 5 uL of the supernatant were transferred to 200 uL of 1:1 acetonitrile/water with 0.1% acetic acid for HPLC analysis according to Table 5-2 and 17-1. See FIG. 5.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. An engineered ketoreductase variant having at least 85%, 90%, 91%, 92%, 93%, 94%, 95%, 96%, 97%, 98%, 99% or more sequence identity to SEQ ID NO: 4, 6, 80, 104, 172, 186, 194, 252, 270, 272, 286, 328, and/or 330.

2. The engineered ketoreductase variant of claim 1, having at least 85%, 90%, 91%, 92%, 93%, 94%, 95%, 96%, 97%, 98%, 99% or more sequence identity to SEQ ID NO: 4, and at least one substitution or substitution set at one or more positions selected from positions 12, 21, 87, 93, 97, 110, 145, 148, 152, 153, 194, 196, 197, 200, 206, 212, and 226, wherein said positions are numbered with reference to SEQ ID NO: 4.

3. The engineered ketoreductase variant of claim 1, having at least 85%, 90%, 91%, 92%, 93%, 94%, 95%, 96%, 97%, 98%, 99% or more sequence identity to SEQ ID NO: 6, and at least one substitution or substitution set selected from 12/110/145/152, 12/145, 87/110/145, 87/110/145/194, 87/145/194, 110, 110/145/152/197, 110/145/194, 145, 145/152, 145/197/226, and 152, wherein said positions are numbered with reference to SEQ ID NO: 6.

4. The engineered ketoreductase variant of claim 1, having at least 85%, 90%, 91%, 92%, 93%, 94%, 95%, 96%, 97%, 98%, 99% or more sequence identity to SEQ ID NO: 80, and at least one substitution or substitution set selected from 17, 21, 46, 56, 72, 79, 95, 101, 110, 152, 162, 190, 198, 210, 211, and 227, wherein said positions are numbered with reference to SEQ ID NO: 80.

5. The engineered ketoreductase variant of claim 1, having at least 85%, 90%, 91%, 92%, 93%, 94%, 95%, 96%, 97%, 98%, 99% or more sequence identity to SEQ ID NO: 80, and at least one substitution or substitution set selected from 17, 79, 157, 159, 190/191/194, 190/194, 191/194, 194, 198, and 211, wherein said positions are numbered with reference to SEQ ID NO: 80.

6. The engineered ketoreductase variant of claim 1, having at least 85%, 90%, 91%, 92%, 93%, 94%, 95%, 96%, 97%, 98%, 99% or more sequence identity to SEQ ID NO: 104, and at least one substitution or substitution set selected from 17/46/190, 17/46/198/211, 17/96/194/198, 17/190/198, 46/190/194/198, and 46/194/198, wherein said positions are numbered with reference to SEQ ID NO: 104.

7. The engineered ketoreductase variant of claim 1, having at least 85%, 90%, 91%, 92%, 93%, 94%, 95%, 96%, 97%, 98%, 99% or more sequence identity to SEQ ID NO: 172, and at least one substitution or substitution set selected from 45, 101, 179, 194, 204, 226, and 231, wherein said positions are numbered with reference to SEQ ID NO: 172.

8. The engineered ketoreductase variant of claim 1, having at least 85%, 90%, 91%, 92%, 93%, 94%, 95%, 96%, 97%, 98%, 99% or more sequence identity to SEQ ID NO: 186, and at least one substitution or substitution set selected from 95/96/97/150/153/205, 95/96/150/153/205/206/211/249, 95/97/143/145/150/153/202/205, 95/97/143/145/150/153/249, 95/97/150/153, 95/97/150/153/202/205/206, 95/150/153/205/206/211, 95/150/153/205/211, 95/150/153/206/249, 96/150/153, 96/150/153/206, 97/150/153, 97/150/153/205, 97/150/153/205/211, 97/150/153/206, 143/144/145/150/153/202/205/249, 143/145/150/153, 144/145/150/153/205/206, 144/150/153, 144/150/153/202/205/206, 145/150/153/206/249, 145/153/211, 150/153/202/206/249, 150/153/205/211, 150/153/206/211, 150/153/211, and 150/153/249, wherein said positions are numbered with reference to SEQ ID NO: 186.

9. The engineered ketoreductase variant of claim 1, having at least 85%, 90%, 91%, 92%, 93%, 94%, 95%, 96%, 97%, 98%, 99% or more sequence identity to SEQ ID NO: 186, and at least one substitution or substitution set selected from 7/147, 103/147, 110, 110/179/194, 147, and 249, wherein said positions are numbered with reference to SEQ ID NO: 186.

10. The engineered ketoreductase variant of claim 1, having at least 85%, 90%, 91%, 92%, 93%, 94%, 95%, 96%, 97%, 98%, 99% or more sequence identity to SEQ ID NO: 194, and at least one substitution or substitution set selected from 7/12/54/110/150/153/194/205/211/249, 12/54/72/110/150/152/153/194/205/211/249, 12/72/101/103/110/152/249, 12/72/110/147/152/204, 45/54/72/110/152/194/204, 72/110/147/150/152/153/194/205/211/249, and 110/150/153/179/194/205/211/249, wherein said positions are numbered with reference to SEQ ID NO: 194.

11. The engineered ketoreductase variant of claim 1, having at least 85%, 90%, 91%, 92%, 93%, 94%, 95%, 96%, 97%, 98%, 99% or more sequence identity to SEQ ID NO: 252, and at least one substitution or substitution set selected from 7/12/54/179/249, 7/152, 12/54/72/152/179/249, 40, 54/72, 72/147/152/179/249, and 249, wherein said positions are numbered with reference to SEQ ID NO: 252.

12. The engineered ketoreductase variant of claim 1, having at least 85%, 90%, 91%, 92%, 93%, 94%, 95%, 96%, 97%, 98%, 99% or more sequence identity to SEQ ID NO: 270, and at least one substitution or substitution set selected from 92/93, 150/152, 150/152/153, and 194/195, wherein said positions are numbered with reference to SEQ ID NO: 270.

13. The engineered ketoreductase variant of claim 1, having at least 85%, 90%, 91%, 92%, 93%, 94%, 95%, 96%, 97%, 98%, 99% or more sequence identity to SEQ ID NO: 272, and at least one substitution or substitution set selected from 92/93/95, 93, 93/95, 93/95/109, 93/95/109/114, 93/95/114, 93/109/114, and 114, wherein said positions are numbered with reference to SEQ ID NO: 272.

14. The engineered ketoreductase variant of claim 1, having at least 85%, 90%, 91%, 92%, 93%, 94%, 95%, 96%, 97%, 98%, 99% or more sequence identity to SEQ ID NO: 286, and at least one substitution or substitution set selected from 12/45/72/109/249, 12/45/93/249, 12/45/249, 12/109/249, 45/72/249, 45/109/249, 45/249, 96, and 145/150, wherein said positions are numbered with reference to SEQ ID NO: 286.

15. The engineered ketoreductase variant of claim 1, having at least 85%, 90%, 91%, 92%, 93%, 94%, 95%, 96%, 97%, 98%, 99% or more sequence identity to SEQ ID NO: 328, and at least one substitution or substitution set selected from 150, 150/151, 150/195, and 195, wherein said positions are numbered with reference to SEQ ID NO: 328.

16. The engineered ketoreductase variant of claim 1, having at least 85%, 90%, 91%, 92%, 93%, 94%, 95%, 96%, 97%, 98%, 99% or more sequence identity to SEQ ID NO: 330, and at least one substitution or substitution set selected from 12/72/109/195, 17/73/200, 17/115, 68/72/101/152/205, 68/72/124, 68/72/124/152, 68/101/124/152/205, 68/124/205, 72/109/152/195, 72/109/195, 72/152, 72/152/195, 72/195, 73, 73/147, 79, 93, 93/95/145/195, 93/109/114/145/195, 93/195, 95/195, 96/108/147/200, 96/194/200, 101/205, 145/195, 147, 147/200, 192, 194, 194/200, 195, 198, and 200, wherein said positions are numbered with reference to SEQ ID NO: 330.

17-18. (canceled)

19. The engineered ketoreductase variant of claim 1, comprising a polypeptide sequence set forth in SEQ ID NO: 6, 80, 104, 172, 186, 194, 252, 270, 272, 286, 328, and/or 330.

20. (canceled)

21. The engineered ketoreductase variant of claim 1, wherein said engineered ketoreductase comprises a polypeptide sequence selected from the even-numbered sequences set forth in SEQ ID NOS: 6 to 412.

22. (canceled)

23. The engineered ketoreductase variant of claim 1 which is capable of converting one or more iso-α-acid substrates to one or more corresponding dihydro-(rho)-iso-α-acid products with at least 10-fold the activity of the reference polypeptide of SEQ ID NO:4.

24. The engineered ketoreductase variant of claim 1, wherein said engineered ketoreductase comprises an improved property as compared to the ketoreductase of SEQ ID NO: 4.

25. The engineered ketoreductase variant of claim 24, wherein the improved property comprises improved activity converting iso-α-acids to the corresponding dihydro-(rho)-iso-α-acids as compared to the ketoreductase of SEQ ID NO: 4.

26. The engineered ketoreductase variant of claim 24, wherein the improved property comprises improved activity at high substrate concentrations as compared to the ketoreductase of SEQ ID NO: 4.

27. The engineered ketoreductase variant of claim 24, wherein the improved property comprises improved activity at low cofactor concentrations as compared to the ketoreductase of SEQ ID NO: 4.

28. An engineered polynucleotide sequence encoding the engineered ketoreductase variant of claim 1.

29. The engineered polynucleotide sequence of claim 28, wherein said sequence comprises a polynucleotide sequence that is at least 85%, 90%, 91%, 92%, 93%, 94%, 95%, 96%, 97%, 98%, 99% or more identical to a sequence selected from the odd-numbered sequences set forth in SEQ ID NOS: 5 to 411.

30. A vector comprising the engineered polynucleotide sequence of claim 28.

31. The vector of claim 30, further comprising at least one control sequence.

32. The vector of claim 30, wherein said vector comprises SEQ ID NO: 413 or 414.

33. A host cell comprising the vector of claim 30 .

34. A method for producing the engineered ketoreductase variant of claim 1, comprising culturing a host cell under conditions that said engineered ketoreductase variant is produced by said host cell.

35. The method of claim 34, further comprising the step of recovering said engineered ketoreductase variant produced by said host cell.

36. The method of claim 34, wherein the engineered ketoreductase variant is produced by a host cell comprising the vector of SEQ ID NO: 413 and/or 414.

37. A composition comprising at least one engineered ketoreductase variant provided in claim 1.
